(19) United States
(12) Patent Application Publication
(10) Pub. No.: US 2025/0195583 A1
(43) Pub. Date: Jun. 19, 2025

(54) LAMB STOMACH EXTRACT VITAMIN B12 PREPARATION COMPOSITION AND THE USE IN THE TREATMENT OF GASTRIC DISEASE THEREOF

(71) Applicant: Xinmin ZHAO, Urumqi (CN)

(72) Inventor: Xinmin ZHAO, Urumqi (CN)

(21) Appl. No.: 18/847,276

(22) PCT Filed: Mar. 14, 2022
(86) PCT No.: PCT/CN2022/080776

§ 371 Date: Sep. 16, 2024

(51) Int. Cl. A61K 35/38; A61K 31/714; A61P 1/04; A61P 35/00

(52) U.S. Cl. CPC A61K 35/38; A61K 31/714; A61P 1/04; A61P 35/00

(57) ABSTRACT

A lamb stomach extract Vitamin B₁₂ preparation composition composed of lamb stomach extract and Vitamin B₁₂ is provided. The lamb stomach extract has a chymosin activity of >40 U/g and a pepsin activity of >15 U/g, and the weight ratio of the lamb stomach extract and Vitamin B₁₂ is 100000:0.5-5, which is useful in the treatment of moderate-to-severe chronic atrophic gastritis, intestinal metaplasia reversal, and gastric cancer prevention. Lamb stomach extract and Vitamin B₁₂ produced synergistic or synergistic effects in specific ratios.

### CROSS REFERENCE TO THE RELATED APPLICATIONS

This application is the national phase entry of International Application No. PCT/CN2022/080776, filed on Mar. 14, 2022, the entire contents of which are incorporated herein by reference.

### TECHNICAL FIELD

The present invention belongs to the pharmaceutical field, and is related to the use of a lamb stomach extract Vitamin B₁₂ preparation composition in the treatment of moderate to severe chronic atrophic gastritis, reversal of intestinal metaplasia and prevention of gastric cancer.

### BACKGROUND

Chronic gastritis is the most common type of digestive disease. Chronic atrophic gastritis is a type of chronic gastritis, accounting for about ⅓ of the chronic gastritis. Clinically, some patients with chronic atrophic gastritis may have no obvious symptoms. However, most patients may have epigastric burning pain, distension, dull pain or fullness, loss of appetite, nausea, belching and other symptoms, especially after meal. In severe cases, there may be emaciation, anemia, brittle nails, tongue inflammation or atrophy of the tongue papilla.

Chronic atrophic gastritis (CAG) is characterized by the reduction of gastric mucosal glands as the main pathological feature, which is characterized by atrophy of gastric glands, thinning of mucosa, thickening of mucosal muscular layer and accompanied by intestinal epithelial metaplasia and atypical metaplasia. Its diagnosis mainly relies on gastroscopic findings and pathological findings of gastric mucosal biopsy.

The disease progression cycle from normal gastric mucosa to gastric cancer is as follows: normal gastric mucosa→non-atrophic gastritis→atrophic gastritis→intestinal metaplasia→intraepithelial neoplasia→gastric cancer. According to the above disease progression, gastric diseases can be divided into non-atrophic gastritis, atrophic gastritis, intestinal metaplasia, intraepithelial neoplasia, and gastric cancer. There are no effective therapeutic drugs for the reversal of atrophic gastritis, intestinal metaplasia, and intraepithelial neoplasia.

### SUMMARY

In order to solve the above problems, it is an object of the present invention to provide a lamb stomach extract Vitamin B₁₂ preparation composition.

Another object of the present invention is to provide a use of the above lamb stomach extract Vitamin B₁₂ preparation composition.

In order to achieve the above objects, the present invention provides a lamb stomach extract Vitamin B₁₂ preparation composition, which is composed of lamb stomach extract and Vitamin B₁₂, wherein the lamb stomach extract has a chymosin activity of >40 U/g and a pepsin activity of >15 U/g, and wherein the weight ratio of the lamb stomach extract and Vitamin B₁₂ is 100000:0.5-5.

Preferably, the weight ratio of the lamb stomach extract and Vitamin B₁₂ is 100000:1-4.5.

More preferably, the weight ratio of the lamb stomach extract and Vitamin B₁₂ is 100000:1, 100000:2, 100000:3, 100000:4 or 100000:4.5.

Preferably, the chymosin activity is >45 U/g and the pepsin activity is >20 U/g; or the chymosin activity is >55 U/g and the pepsin activity is >20 U/g.

Preferably, the lamb stomach extract protein has a relative molecular weight of 10,000 to 45,000.

Preferably, the ratio of the chymosin activity to the pepsin activity is from 2.5 to 5.1.

The composition as mentioned above may further comprise a pharmaceutically acceptable additive. Preferably, the pharmaceutically acceptable additive is one or more of an excipient, a binder, a diluent, a flow aid, a flavoring agent, an antioxidant, a bacteriostatic agent.

As mentioned above, the dosage form of the preparation composition is a capsule, tablet, suspension or granule.

The present invention also provides the use of the lamb stomach extract Vitamin B₁₂ preparation composition in the manufacture of a medicament for reversing atrophy of the gastric mucosa.

The present invention also provides the use of lamb stomach extract Vitamin B₁₂ preparation composition in the manufacture of a medicament for the treatment of moderate or severe chronic atrophic gastritis.

The present invention also provides the use of the lamb stomach extract Vitamin B₁₂ preparation composition in the manufacture of a medicament for the treatment of intestinal metaplasia.

As described above, the intestinal metaplasia is the intestinal metaplasia accompanied with chronic atrophic gastritis.

The present invention also mentions the use of lamb stomach extract Vitamin B₁₂ preparation composition in the manufacture of a medicament for the treatment of gastric intraepithelial neoplasia.

The present invention also provides the use of lamb stomach extract Vitamin B₁₂ preparation composition in the manufacture of a medicament for the prevention of gastric cancer.

As mentioned above, the treatment is an effective treatment.

As mentioned above, the treatment is a reversal treatment.

As mentioned above, the subject for administration is a patient who is Helicobacter pylori-negative.

As mentioned above, the treatment, reversal, and prevention are carried out through the antioxidant pathway.

As mentioned above, the treatment, reversal, and prevention are carried out by regulating intestinal flora dysbiosis.

As mentioned above, the preparation composition is administered to the patient at 330 U, 3 times/d.

The ratio of the dosage in the lamb stomach extract and Vitamin B₁₂ preparation composition provided herein has a direct impact on the therapeutic effect. Specifically, the lamb stomach extract and Vitamin B₁₂ in specific ratios produce a synergistic or enhanced effect. Synergistic effects are observed in the range of 100,000:4-100,000:1 weight ratio of lamb stomach extract to Vitamin B₁₂ as compared to lamb stomach extract or Vitamin B₁₂ alone. Compared with the 100,000:4 weight ratio, there is an advantage in controlling residual basal mucosal congestion at lower weight ratios of 100,000:1 for the combination of lamb stomach extract and Vitamin B₁₂. Further reduction in the weight ratio of Vitamin B₁₂ to 100000:0.2 did not result in synergistic effect of the preparation composition.

According to the lamb stomach extract Vitamin B₁₂ preparation composition of the present invention, the preparation composition comprises a lamb stomach extract having a chymosin activity of not less than 45 U/g and a pepsin activity of not less than 20 U/g; or a chymosin activity of not less than 55 U/g and a pepsin activity of not less than 20 U/g.

The beneficial effects of the present invention are as follows.

The present invention provides a lamb stomach extract Vitamin B₁₂ preparation composition, which can effectively reverse gastric mucosal atrophy, intestinal metaplasia and intraepithelial neoplasia, can significantly reduce the OLGA and OLGIM stages of patients with severe CAG, and can effectively reduce the risk of gastric cancer.

### BRIEF DESCRIPTION OF THE DRAWINGS

FIG. 1 shows a graph of the results of the comparison of the gastric fluid pH of rats in the negative control, administered and non-administered groups of the lamb stomach extract Vitamin B₁₂ preparation provided by the present invention.

FIGS. 2A-2F show the graphs of pathological changes in the gastric mucosa of rats in the negative control, administered and non-administered groups of the lamb stomach extract Vitamin B₁₂ preparation provided by the present invention.

FIG. 3 shows the effective rate of Vitamin B₁₂ preparation of lamb stomach extract provided by the present invention on chronic inflammation in various parts of patients with chronic atrophic gastritis.

FIG. 4 shows the incidence of reversal of chronic inflammation at various sites/effective rate of inflammatory activity at various sites in patients with chronic atrophic gastritis by the lamb stomach Vitamin B₁₂ preparation composition extract provided by the present invention.

FIG. 5 shows the effective rate of atrophy in various parts of patients with chronic atrophic gastritis by the lamb stomach extract Vitamin B₁₂ preparation composition provided by the present invention.

FIG. 6 shows the incidence of reversal of atrophy at various sites in patients with chronic atrophic gastritis by the lamb stomach extract Vitamin B₁₂ preparation composition provided by the present invention.

FIG. 7 shows the effective rate of intestinal epithelial metaplasia at various sites in patients with chronic atrophic gastritis by the lamb stomach extract Vitamin B₁₂ preparation composition provided by the present invention.

FIG. 8 shows the incidence of reversal of intestinal epithelial metaplasia at various sites in patients with chronic atrophic gastritis by the lamb stomach extract Vitamin B₁₂ preparation composition provided by the present invention.

FIG. 9 shows a comprehensive evaluation of the effective rate of the lamb stomach extract Vitamin B₁₂ preparation provided by the present invention on gastric chronic inflammation, atrophy and intestinal metaplasia in patients with chronic atrophic gastritis.

FIG. 10 shows a comprehensive evaluation of the incidence of gastric chronic inflammation, atrophy and intestinal metaplasia reversal in patients with chronic atrophic gastritis by the lamb stomach extract Vitamin B₁₂ preparation composition provided by the present invention.

FIGS. 11A-11B show the patient's gastroscopy findings on May 13, 2019, wherein FIG. 11A shows gastric antrum and FIG. 11B shows gastric body.

FIGS. 12A-12B show the patient's biopsy histopathology findings on May 13, 2019, with hematoxylin-eosin staining at low magnification, wherein FIG. 12A shows gastric antrum and FIG. 12B shows gastric body.

FIGS. 13A-13B show the patient's gastroscopy findings on Dec. 11, 2019, wherein FIG. 13A shows gastric antrum and FIG. 13B shows gastric body.

FIGS. 14A-14B show the patient's biopsy histopathology findings on Dec. 11, 2019, with hematoxylin-eosin staining at low magnification, wherein FIG. 14A shows gastric antrum and FIG. 14B shows gastric body.

FIGS. 15A-15B show the patient's gastroscopic findings on Mar. 30, 2021, wherein FIG. 15A shows gastric antrum and FIG. 15B shows gastric body.

FIGS. 16A-16B show the patient's biopsy histopathology findings on Mar. 30, 2021, with hematoxylin-eosin staining at low magnification, wherein FIG. 16A shows gastric antrum and FIG. 16B shows gastric body.

### DETAILED DESCRIPTION OF THE EMBODIMENTS

The following embodiments of the present invention will be mentioned in a detailed and comprehensive manner, so that the advantages and features of the present invention can be more easily understood by those skilled in the art, and so that the scope of protection of the present invention can be more clearly and explicitly defined. It should be understood that the specific embodiments mentioned herein are only for the purpose of explaining the present invention and are not intended to limit the present invention.

In the lamb stomach extract Vitamin B₁₂ oral preparation or granules, the main ingredients of were highly active organisms extracted from the 4th stomach (wrinkled stomach) of sheep in Tianshan Mountain, Xinjiang (containing a variety of biologically active ingredients such as chymosin, pepsin, mucin, bifidus factor) and Vitamin B₁₂.

### Example 1 Extraction and Isolation and Purification of Enzymes from Wrinkled Stomach in Lamb Stomach Extracts

Lamb stomach extract was degreased by petroleum ether and then extracted with 0.9% sodium chloride solution under low temperature with ultrasonic assistance. The extract comprised 0.5 mol/L hydrochloric acid solution at pH 4.6. The sample was sequentially precipitated with 30%, 50%, and 90% saturated ammonium sulfate. Finally, 90% saturated ammonium sulfate precipitate was taken for measurement of total enzyme activity. The precipitates were dissolved in deionized water, and desalted with a dialysis bag, and then separated and purified with diethylaminoethyl (DEAE) cellulose 52 anion exchange resin. The samples were gradiently eluted with equilibrium solution (0.05 mol/L phosphate buffer at pH 5.7) and equilibrium solution containing 0.1, 0.2, 0.3, 0.4, and 0.5 mol/L NaCl, and the eluates were detected at 280 nm UV wavelength. The effluent was collected at a rate of 10 min/tube, and the chymosin activity was determined. The 0.4 and 0.5 mol/L NaCl solution elution sites (named F6 and F7) possessed chymosin activity, and were further separated and purified by high-pressure liquid chromatography (HPLC) on a gel column with 0.9% NaCl solution as the eluent at a flow rate of 0.4 mL/min. Protein absorption was detected at dual wavelengths of 212 and 280 nm and the samples were collected. The chymosin activity of the samples was determined by the Arima method and the pepsin activity was determined by the Mazorra Manzano method. The samples were subjected to sodium dodecyl sulfate with 15% separating gel polyacrylamide gel electrophoresis (sodium dodecyl sulfate polyacrylamide gel electrophoresis, SDS-PAGE). LC MS/MS analysis was performed on F6-2, which was enzymatically cleaved into small molecule peptides by trypsin, and the peptides were subjected to LC MS/MS analysis, and the analyzed data were identified using the similarity search tool of the SIB ExPASy bioinformatics database. The enzyme activities and yields in different isolation and purification stages of the lamb stomach extract are shown in Table 1. The chymosin activity of the lamb stomach extract was (691.2±5.16) U/g, and after one step purification by saturated ammonium sulfate precipitation the chymosin activity of the chymosin enzyme was increased by 26.5-fold to (18341.61±4.1) U/g. The chromatographic results of isolation and purification by the DEAE-cellulose 52 anion-exchange columns of the total enzyme activity sites showed that, among the seven eluted sites, F6 and F7 had chymosin activity and F7 also had pepsin activity. The isolated and purified chymosin F6-2 and F7-2 had high chymosin activity and low casein hydrolysis activity, and pepsin had bovine serum albumin hydrolysis activity, and that F7-1 had the property as measured. The HPLC separation and purification profiles showed that, F6-2 and F7-2 were chymosin, and F7-1 was pepsin, and the enzyme activities were (27,557.1±4.8) U/g, (17,532.6±5.1) U/g, and (17728.15±5.3) U/g, respectively. The relative molecular weights of F6-2, F7-2 and F7-1 were all in the range of 35 000 to 40 000. The results of LC-MS/MS (nano LC-QE) analysis and database comparison of the chymosin are shown in Table 2, and the relative molecular weight of the chymosin F6-2 was 40,392.88 with an isoelectric point of 4.58.

TABLE 1
Enzyme activities and yields in different stages of isolation and purification of lamb stomach extracts

| Purification | | Chymosin | Proten hydrolysis activity U/g | | Purification | Yield |
|---|---|---|---|---|---|---|
| Method | | activity (U/g) | Casein | BSA | Factor | (%) |
| API | | 691.20 ± 5.16^{h} | — | 1016.59 ± 7.80^{h} | 1.00 | 100 |
| Degreased API | | 912.01 ± 8.75^{g} | 5.20 ± 5.30^{h} | 1804.00 ± 6.10ⁱ | 1.31 | 73 |
| Extract | | 6747.04 ± 6.80^{f} | 33.02 ± 7.80^{d} | 6404.54 ± 5.30^{d} | 9.76 | 6.45 |
| Total enzyme active site | | 18341.61 ± 4.10^{e} | 67.75 ± 3.10^{c} | 13026.93 ± 8.10^{c} | 26.50 | 0.9 |
| DEAE- | F6 | 25557.10 ± 4.20^{b} | 27.90 ± 5.80^{e} | 5446.04 ± 4.80^{e} | 36.90 | 0.223 |
| cellose 52 | F7 | 15552.60 ± 5.10^{d} | 84.86 ± 4.20^{b} | 15728.15 ± 5.30^{b} | | |
| | F6-2 | 27557.10 ± 4.80^{a} | 25.90 ± 3.40^{e, f} | 4346.04 ± 4.80^{f} | | |
| HPLC-TSKgel | F7-2 | 17532.60 ± 5.10^{c} | 21.90 ± 3.80^{g} | 4846.04 ± 4.80^{g} | 39.50 | 0.089 |
| | F7-1 | — | 89.86 ± 4.20^{a} | 17728.15 ± 5.30^{a} | | |

Note:
“—” means no such value. Different letters in the same column indicate a statistically different difference between the two (P < 0.05).
DEAE is diethylaminoethyl;
HPLC-TSKgel is high-performance liquid chromatography gel column;
BSA is bovine serum albumin

TABLE 2
Results of liquid chromatography-tandem mass spectrometry identification of chymosin F6-2

| | Percentage Of Structural | Relative | |
|---|---|---|---|
| | Domains Of Chymosin Activity | Molecular | Isoelectric |
| Reference | (%) | Weight | Point |
| tr\|A0A3Q1MMC7\|A0A3Q1MMC7_BOVIN | 2.45 | 40 472.19 | 5.14 |
| tr\|C0LTP1\|C0LTP1_BOVIN | 2.36 | 42 179.18 | 4.90 |
| tr\|Q8SPG7\|Q8SPG7_BOVIN | 3.41 | 29 020.37 | 4.61 |
| tr\|Q8SPG5\|Q8SPG5_BOVIN | 3.98 | 24 649.56 | 4.63 |
| tr\|Q28075\|Q28075_BOVIN | 3.72 | 26 683.57 | 4.03 |
| tr\|A0A6P5BJK1\|A0A6P5BJK1_BOSIN | 2.36 | 42 237.22 | 4.83 |
| tr\|L8IT38\|L8IT38_9CETA | 2.36 | 42 184.20 | 4.90 |
| tr\|W5QDV4\|W5QDV4_SHEEP | 2.36 | 42 086.14 | 4.80 |
| tr\|M4LK61\|M4LK61_BOSMU | 2.36 | 42 207.19 | 4.83 |
| sp\|P00794\|CHYM_BOVIN | 2.36 | 42 179.18 | 4.90 |
| sp\|P18276\|CHYM_SHEEP | 2.36 | 42 074.09 | 4.80 |
| tr\|A0A6J0WDJ8\|A0A6J0WDJ8 | 2.36 | 42 186.44 | 5.08 |
| tr\|A0A6P3GQP2\|A0A6P3GQP2 | 2.36 | 42 198.23 | 4.90 |
| tr\|Q8SPG8\|Q8SPG8_BOVIN | 3.03 | 32 763.56 | 4.49 |
| tr\|Q8SPG6\|Q8SPG6_BOVIN | 3.47 | 28 392.76 | 4.49 |
| tr\|Q8SPH0\|Q8SPH0_BOVIN | 2.66 | 37 221.56 | 4.86 |
| tr\|Q8SPG9\|Q8SPG9_BOVIN | 2.95 | 33 478.36 | 5.10 |
| tr\|Q8SPH1\|Q8SPH1_BOVIN | 2.62 | 37 849.17 | 5.02 |
| F6-2 | 2.43 | 40 392.88 | 4.58 |

### Example 2 Analysis of Antioxidant Active Components of Lamb Stomach Extracts

Lamb stomach extract was acid hydrolyzed and subjected to DNFB derivatization, and their amino acid composition was analyzed using HPLC Diamonsil AAA amino acid analytical columns. The APIs were extracted sequentially with water, 0.1 mol/L phosphate buffer at pH 7.0, and 0.01 mol/L aqueous ammonium bicarbonate to dissolve the APIs completely. Extracts were desalted, freeze-dried, and stored at −20° C. for stock. The three extracts were subjected to 1,1-diphenyl-2-picrylhydrazyl radical 2,2-diphenyl-1-(2,4,6-trinitrophenyl)hydrazyl [DPPH] and 2,2′-azinobis-(3-ethylbenzthiazoline-6-sulphonate) [ABTS] free radical in vitro antioxidation activity measurement using the method reported in literature [13]. A 15% 15% separating gel was used for SDS-PAGE to analyze the relative molecular weight range of proteins in the three extracts.

The results of amino acid composition analysis of the lamb stomach extracts are shown in Table 3, and the hydrophilic amino acids directly involved in the antioxidant reaction process accounted for 33.04% of the total amino acid content. At a concentration of 5 g/L for the samples, the in vitro antioxidant activity ABTS scavenging activity of the three extracts extracted from water, phosphate buffer and ammonium bicarbonate solution of the API was (37.08±0.45)%, (23.20±0.78)% and (62.80±0.74)%, respectively, and DPPH scavenging activity was (57.87±0.55)%, (5.03±0.25)% and (26.67±0.21)%, respectively. The comparison results of antioxidant activity showed that the water and ammonium bicarbonate extraction sites possessed higher ABTS and DPPH scavenging capacities, and had higher antioxidant activity as compared to phosphate buffer extracts. The SDS-PAGE results of the three extracts showed that the relative molecular weight of the proteins in the APIs ranged from 10,000 to 40,000.

TABLE 3
Amino acid composition of lamb stomach extracts

| | Amino Acid | Content |
|---|---|---|
| No. | Abbreviation | g/kg |
| 1 | Asp | 8.36 |
| 2 | Glu | 1.33 |
| 3 | Ser | 4.79 |
| 4 | Gly | 4.99 |
| 5 | His | 1.84 |
| 6 | Arg | 9.91 |
| 7 | Thr | 4.0 |
| 8 | Ala | 4.581 |
| 9 | Pro | 5.14 |
| 10 | Tyr | 5.30 |
| 11 | Val | 4.205 |
| 12 | Ile | 3.731 |
| 13 | Leu | 7.281 |
| 14 | Phe | 3.751 |
| 15 | Lys | 6.204 |
| 16 | Met | 1.022 |

Analysis of Crude Extracts of Lamb Stomach Glycoproteins and their Probiotic Activities

The results in Table 4 showed that the crude extract of glycoproteins showed growth-promoting effects on Bifidobacterium adolescentis, Lactobacillus delbrueckii Bulgaria subspecies, and Enterococcus faecalis, with statistically significant differences in the growth-promoting effects on Lactobacillus delbrueckii Bulgaria subspecies and Enterococcus faecalis (all P<0.05). The elution peaks of protein and sugar in the elution curve of the chromatogram overlapped with each other for glycoproteins, and GP was the glycoprotein purified by Sephadex G-50, and GP-1 was the glycoprotein isolated and purified by DEAE-cellulose 52. The total sugar content of GP-1 was 70.24%, and the protein content was 16.39%; the polysaccharide chain consisted of two monosaccharides, lactose and glucose, and the amino acid composition was as shown in Table 5.

TABLE 4
Results of pro-growth activity of intestinal probiotics of glycoproteins

| | | Sample | | |
|---|---|---|---|---|
| | | Concentration | Bacterial | Comparison with control |
| Strains | Tested Group | (mg/mL) | (pcs/mL) | (p value ^{a}) |
| Bifidobacterium | Negative Control | 0 | 9.8 ± 0.34 × 10³ | |
| adolescentis | FOS | 10 | 5.9 ± 0.00 × 10³ | 0.1161 |
| | Crude | 10 | 1.17 ± 0.59 × 10⁴ | 0.6413 |
| | Glycoprotein | | | |
| Lactobacillus | Negative Control | 0 | 1.81 ± 0.50 × 10⁶ | |
| delbrueckii | FOS | 10 | 1.62 ± 0.26 × 10⁶ | 0.5882 |
| Bulgaria subspecies | Crude | 10 | 16.93 ± 0.79 × 10⁶ | 0.0011 |
| | Glycoprotein | | | |
| Enterococcus | Negative Control | 0 | 1.94 ± 0.02 × 10⁶ | |
| faecalis | Crude | 25 | 5.68 ± 0.30 × 10⁶ | <0.0001 |
| | Glycoprotein | | | |

Note:
^{a} Comparison with negative control

TABLE 5
Amino acid composition of protein chains in glycoproteins
isolated and purified from diethylaminoethyl cellulose 52

| No. | Amino Acid | Content g/kg |
|---|---|---|
| 1 | Asp | 0.86 |
| 2 | Glu | 1.11 |
| 3 | Ser | 2.25 |
| 4 | Gly | 1.83 |
| 5 | His | 0.87 |
| 6 | Arg | 0.68 |
| 7 | Thr | 2.52 |
| 8 | Ala | 0.73 |
| 9 | Pro | 2.23 |
| 10 | Tyr | 0.45 |
| 11 | Val | 0.98 |
| 12 | Ile | 0.46 |
| 13 | Leu | 0.51 |
| 14 | Phe | 0.29 |
| 15 | Lys | 0.63 |

| | |
|---|---|
| Total Amino Acid | 16.39 |

The antioxidant activity of the three extracts of the API showed that all the three extracts had antioxidant activity, where the water extract and ammonium bicarbonate extract sites showed higher antioxidant activity, which may be the main component of the drug that plays a role as a pharmacodynamic agent in the treatment and prevention of gastric ulcers, chronic atrophic gastritis, and gastric cancer.

### Example 3 Sub-Acute Toxicity Test for Chymosin and Pepsin Activity in Lamb Stomach Extract Vitamin B₁₂ Preparation

### I. Materials and Methods

1. Animals: 30 healthy rats (collected from the animal house and fed for one week before being used in the experiment) weighing 115-195 grams. There were three groups of 10 rats each.
2. Sample: Lamb stomach extract Vitamin B₁₂ preparation
3. Methods: Three groups of animals were divided into a control group, a low dose group and a high dose group.

The control group was orally administered with 1.5 ml of saline daily for 30 days.

The low dose group was orally administered daily at 500 mg/kg body weight for 30 days with the samples prepared as 1.5 ml suspension in saline.

The high-dose group was orally administered daily at 1500 mg/kg body weight for 30 days with the samples prepared as 1.5 ml suspension in saline.
4. Observation items: Before and 30 days after the administration of the drug, the activity of serum glutamic pyruvic enzyme (SGPT) and the content of serum non-protein nitrogen of the animals were measured respectively. The pathological sections of the liver, spleen, heart, kidney, lesser, small intestines, stomach and other organs of the animals after 30 days of the administration of the drug were examined, in order to observe the effect of the drug on the function of the liver and kidney, and the changes of the various organs of the animals.

### II. Determination of Serum Glutamine Transaminase (SGPT) Activity

Serum glutamate transhydrogenase is highest in hepatocytes. When certain drugs cause liver damage or are in the acute stage of viral hepatitis, the hepatocytes are damaged and the enzyme overflows into the bloodstream, which can lead to a significant increase in serum glutamate transaminase activity. Therefore, changes in serum glutamine transaminase activity can be used as one of the important indicators of sub-acute poisoning by drugs and liver lesions.

### Measurement Methods

### (I) Reagents:

1. Pyruvic acid standard solution: 23.5 mg of sodium pyruvate, with 0.1M pH7.4 phosphate buffer solution to dissolve and fix the volume to 100 ml.
2. Substrate solution: 87.6 mg of ketoglutaric acid and 5.34 g of dl-alanine, first dissolved in 90 ml of 0.Im, pH7.4 phosphate buffer, adjusted to pH7.4 with 20%, NaOH solution, and then diluted to 300 ml of the above phosphate buffer, stored in the refrigerator, can be used for a week.
3. 2,4-Dinitrophenylhydrazine solution: 19 8 mg, 2,4-Dinitrophenylhydrazine, dissolved in 8.0 ml of concentrated hydrochloric acid, transferred to a 100 ml volumetric flask, and fixed to scale with distilled water.
4. 0.4 N NaOH solution
5. 0.1 M, pH7. 4 phosphate buffer: 2.69 g KH₂PO₄ and 13.97 g K₂HPO₄, dissolved with water, transferred to a 1000 ml volumetric flask and diluted to scale with distilled water.

### (II) Preparation of Standard Curves:

6 dry and clean test tubes were taken and numbered 0, 1, 2, 3, 4, 5. Each tube was added with 0, 0.05, 0.10, 0.15, 0.20, 0.25 ml of pyruvic acid standard solutions, further added with 0.1M, pH7.4 phosphate buffer 0.25, 0.20, 0.15, 0.10, 0.05, 0 ml, then further added with 0.25 ml of SGPT substrate solution and 0.5 ml of 2,4-dinitrophenyl hydrazine solution for each tube to sufficiently mix. The mixture was held in 37° C. water bath for 20 mins, and then each tube were added with the 0.4N NaOH rye 5.0 ml and mixed, held under room temperature for 10 mins. The 750 spectrophotometer was calibrated for the zero point with distilled water. Using a 200 u microcell, when the light absorption is measured at 520 nm wavelength, the optical density difference between each tube and “0” tube is taken as the ordinate, and the corresponding microgram of pyruvate is taken as the abscissa, and a standard curve was drawn.

### (III) SGPT Activity Measurement

Animal serum preparation: 0.5-1.0 ml of blood was removed from rats with severed tails, and the clot was centrifuged at 3000 rpm for 10 mins, and the serum was taken for spare.

Several dry and clean test tubes were taken, one of which as a control tube. The rest were measurement tubes. Each tube was added with 0.02 ml of corresponding serum by microinjection of stomach, and the measuring tube was added with 0.10 ml of SGPT substrate solution, with sufficient mixing. The mixture was kept warm in water bath at 37° C. for 30 mins, added with 0.10 ml of 2,4-dinitrophenylhydrazine solution to each tube and added with 0.10 ml of SGPT substrate solution to the control tube, with sufficient mixing. The mixture was kept warm in water bath at 37° C. for 20 mins, and then added with 1.0 ml of 0.4N NaOH to each tube. Adjust the zero point with distilled water and photometrically absorb at 520n wavelength. The spectrophotometer was calibrated for the zero point with distilled water. The light absorption was measured at 520 nm wavelength. The microgram of pyruvate was found on the standard curve, and the SGPT activity was calculated according to the following formula.

$$SGPT Activity = ( micrograms in measurement tube - micrograms in control tube ) / ( 2.5 * 0.1 ) ( U / ml serum )$$

Unit Definition: 1 mL of serum interacted with the substrate for 30 min at 37° C. 1 SGPT unit means the production of 2.5 ug of pyruvate. Normal human SGPT values range from 2-40 units.

### III. Determination of Serum Non-Protein Nitrogen Content

Serum non-protein nitrogen is a general term for nitrogen in urea, uric acid, amino acids, ammonia and bilirubin, mainly urea nitrogen. When the serum non-protein nitrogen is significantly elevated, it indicates that the kidney is damaged. Therefore, the determination of serum non-protein nitrogen content can be used to estimate the impact of drug subacute poisoning on the kidney and the degree of renal lesions.

### Measurement Methods

### (I) Reagents

1. 10% sodium tungstate solution
2. ⅔N NaOH solution
3. 1:1 sulfuric acid
4. Nessler reagent: In 500 ml flask, 22.5 g iodine, 30 g ′Kl and water 20 ml were added to dissolve. 30 g of alcohol was added, with sufficient shaking. The mixture was cooled intermittently with water, shaken to the supernatant until the disappearance of the yellow color. The supernatant was poured into 200 ml volumetric flask, diluted to the scale, mixed, added to 975 ml of 2.5N NaOH solution and mixed, stored in a brown bottle, clarified and stocked for use.
5. Standard ammonium sulfate solution (0.06 mg of nitrogen per milliliter): Put pure ammonium sulfate in a dryer for several days, put 0.2834 g in a 10 ml volumetric flask, dilute it to scale, and mix it evenly (at this time, double the original solution to obtain an application solution containing 0.03 mg nitrogen per ml).

### (II) Measurement

1. Preparation of protein-free filtrate: Add 0.7 ml of water, 0.1 ml of serum and 0.1 ml of 10% sodium tungstate into a 5 ml dry and clean centrifuge tube, mix well, then add 0.1 ml of ⅔N H₂SO₄ solution, mix well, stand for 10 minutes, centrifuge at 3000 RPM for 10 minutes, and take the supernatant for later use.
2. Take three hard test tubes, one for measurement, one for reference tube and one for blank tube. Add 0.5 ml of the above protein-free filtrate and 0.1 ml of 1:1 sulfuric acid into the measuring tube, mix them evenly, digest them on an alcohol lamp with low fire until they are colorless, and then cool them. Fix the volume to 5.0 ml, add 0.5 ml of standard ammonium sulfate application solution and 0.1 ml of 1:1 sulfuric acid and 4.4 ml of distilled water into the standard tube. Add 5.0 ml distilled water to the blank tube.

Add 1.5 ml Nessler's reagent to the above three tubes, mix them evenly. The spectrophotometer was calibrated for the zero point with the blank tube at the wavelength of 420 nm. Calculate the non-protein nitrogen content per 100 ml of serum according to the following formula.

$$mg \% = ( Sample tube light absorption ) / ( Reference tube light absorption ) * ( 0.5 * 0.03 ) * 100 / 0.05$$

Normal human 100 ml of blood contains 21-42.5 mg of non-protein nitrogen.

### IV. Result and Discussion

TABLE 6
Comparison of SGPT activity and N—P—N content in the control
group before and after administration of 1.5 ml saline.

| Rat No. | | 1 | 2 | 3 | 4 | 5 | 6 | 7 | 8 | 9 |
|---|---|---|---|---|---|---|---|---|---|---|
| SGPT | Before | 29 | 33 | 27 | 37 | 33 | 48 | 49 | 21 | 33 |
| | administration | | | | | | | | | |
| Activity (u) | After | 22 | 22 | 14 | 26 | 18 | 14 | 20 | 32 | 28 |
| | administration | | | | | | | | | |
| N•P•N Content | Before | 39.1 | 39.1 | 36.5 | 30.1 | 44.0 | 36.6 | 46.6 | 34.4 | 50.0 |
| | administration | | | | | | | | | |
| (mg %) | After | 36.7 | 36.0 | 29.5 | 34.8 | 27.8 | 44.6 | 41.7 | 44.6 | 41.1 |
| | administration | | | | | | | | | |

[Note]
A rat died in the experiment through the gastric lavage.

TABLE 7
Comparison of SGPT activity and NPN content in low dose (500 mg API/kg body weight) group

| Rat No. | | 1 | 2 | 3 | 4 | 5 | 6 | 7 | 8 | 9 | 10 |
|---|---|---|---|---|---|---|---|---|---|---|---|
| SGPT | Before | 30 | 24 | 38 | 32 | 34 | 16 | 20 | 22 | 36 | 18 |
| Activity (U) | administration | | | | | | | | | | |
| | After | 18 | 24 | 14 | 12 | 12 | 10 | 17 | 8 | 10 | 10 |
| | administration | | | | | | | | | | |
| N•P•N Content | Before | 60.5 | 34.9 | 40.0 | 42.4 | 41.1 | 41.3 | 37.5 | 39.8 | 36.2 | 51.8 |
| Mg % | administration | | | | | | | | | | |
| | After | 38.8 | 36.2 | 35.9 | 28.9 | 36.6 | 33.8 | 35.3 | 26.9 | 29.3 | 27.1 |
| | administration | | | | | | | | | | |

TABLE 8
Comparison of SGPT activity and NPN content in
high dose (1500 mg API/kg body weight) group

| Rat No. | | 1 | 2 | 3 | 4 | 5 | 6 | 7 | 8 |
|---|---|---|---|---|---|---|---|---|---|
| SGPT | Before administration | 30 | 36 | 36 | 30 | 36 | 40 | 38 | 40 |
| Activity (U) | After administration | 16 | 20 | 24 | 16 | 24 | 22 | 20 | 12 |
| N•P•N Content | Before administration | 35.1 | 42.3 | 33.2 | 40.5 | 42.8 | 62.0 | 43.5 | 51.5 |
| mg % | After administration | 24.0 | 17.5 | 24.5 | 30.1 | 30.1 | 28.7 | 26.5 | 27.3 |

[Note]
Two rats died in the experiment through the gastric lavage.

TABLE 9
Number of groups

| | Control Group | | | | Low-dose Group | | | | High-dose Group | | | |
|---|---|---|---|---|---|---|---|---|---|---|---|---|
| | Comparison | | | | Comparison | | | | Comparison | | | |
| | before and | | | | before and | | | | before and | | | |
| | after | | Self-comparison | | after | | Self-comparison | | after | | Self-comparison | |
| | Before | After | Standard | Confidence | Before | After | Standard | Confidence | Before | After | Standard | Confidence |
| | admini- | admini- | Error | Interval | admini- | admini- | Error | Interval | admini- | admini- | Error | Interval |
| | stration | stration | S4 | (%) | stration | stration | S4 | (%) | stration | stration | S4 | (%) |
| SGPT | 34.4 ± | 21.8 ± | 0.58 | >99.9 | 27.0 ± | 13.2 ± | 2.8 | >99.9 | 35.8 ± | 18 ± | 1.84 | >99.9 |
| Activity | 9.18 | 6.12 | | | 7.9 | 4.7 | | | 3.9 | 4.47 | | |
| (U) | | | | | | | | | | | | |
| PNP | 39.2 ± | 37.4 ± | 3.1 | >90 | 42.3 ± | 32.9 ± | 2.7 | >99.9 | 43.9 ± | 26.1 ± | 3.2 | >99.9 |
| Content | 6.6 | 6.1 | | | 7.8 | 4.4 | | | 13.6 | 13.0 | | |
| (mg %) | | | | | | | | | | | | |

As can be seen from Tables 6-9, compared with normal rats, before administration, two of the 30 rats had SGPT activity greater than 40 units, and 10 rats had NPN content above 42.5 mg %. However, after administration, all rats had SGPT activity below 40 units. As for the NPN content, after administration, the serum NPN content of all rats was in the normal range, except for two rats in the control group which were above 42.5 mg %. It indicates that the lamb stomach extract Vitamin B₁₂ preparation did not damage the liver and kidney of rats.

### V. Observation of Pathological Sections

After 30 days of drug administration, the rats were put to death, and the liver, lung, heart, spleen, kidney, stomach and a section of small intestine were immediately removed and fixed in 10% formaldehyde solution, and upon sectioning. There was no significant difference between the control group and the drug administration group.

### Example 4 Therapeutic Effect of Lamb Stomach Extract Vitamin B₂ Preparation on Precancerous Lesions of Rat Gastric Mucosa

### Research Method I:

### Drugs for Testing:

The chymosin activity of the lamb stomach extract in the test was 45 U/g, pepsin was 20 U/g, and the relative molecular weight of the protein was 10,000 to 40,000. Vitamin B₁₂ was a U.S.P Vitamin B₁₂.

### Control and Treatment Groups

Animals were distributed as follows:
The control group (A) received only p.o water under the same conditions as the other groups.
Treatment group (B) used a combination of lamb stomach extract: Vitamin B₁₂ with a weight ratio of 100,000:1.
The treatment group (C) used only lamb stomach extract without Vitamin B₁₂.
The treatment group (D) used a combination of lamb stomach extract: Vitamin B₁₂ with a weight ratio of 100,000:0.2.
Treatment group (E) used a combination of lamb stomach extract: Vitamin B₁₂ with a weight ratio of 100,000:4.

Treatment in each group was administered orally at a dose of 400 mg/kg by formulating the test complex into an aqueous suspension for use, with the exception of the group that was treated with Vitamin B₁₂ only under the same conditions. Vitamin B₁₂ was dosed at (4 ug/kg).

Statistical comparisons of weight changes in the different experimental groups were made by applying an ANCOVA analysis for each day's data.

The number of times peptic mucosal ulcers and inflammation appeared in different experimental groups is shown in Table 10. Each group contained four animals.

TABLE 10

| Signs of | Numbers of Lesioned Animals | | | | | |
|---|---|---|---|---|---|---|
| macroscopic changes in | Control | Treatment | Treatment | Treatment | Treatment | Treatment |
| the digestive mucosa | Group A | Group B | Group C | Group D | Group E | Group F |
| Residual basal mucosal | 4 | 1 | 4 | 3 | 3 | 4 |
| congestion | | | | | | |
| Inflammation or ulcer | 4 | 0 | 3 | 2 | 0 | 4 |
| formation of residual basal | | | | | | |
| mucosa | | | | | | |
| Anastomotic congestion | 4 | 2 | 3 | 4 | 1 | 4 |
| Inflammation of the cavity | 4 | 1 | 4 | 4 | 2 | 4 |
| mucosa | | | | | | |
| Ulceration of the mucosa | 3 | 1 | 3 | 3 | 1 | 4 |
| of the cavity or ileum | | | | | | |
| Colon congestion | 4 | 1 | 3 | 3 | 1 | 5 |

As can be seen in Table 6, treatment group B had a significant ameliorative effect on the appearance of gastrointestinal mucosal inflammation. This effect was observed in all aspects of the gastrointestinal tract observed in the autopsy. The treatment group E and group B had the same clinical effect and similar protective effect. A lower number of residual basal mucosal congestion was observed in the treatment group B than in the treatment group E.

According to the comparisons, group C, which was treated only with lamb stomach extract without Vitamin B₁₂, was similar to control group A. Control group A was the same as group F, which was treated only with Vitamin B₁₂. The severity of gastrointestinal damage was the same for groups B and E. The results of groups B and E were the same. For group D, it was only a slight improvement over the results of control group A.

As can be seen from Table 6, both lamb stomach extract and Vitamin B₁₂ should be used. In terms of efficacy, under the same experimental conditions, treatment group C (lamb stomach extract without Vitamin B₁₂) showed no protective effect against phenylbutazone-induced ulcers, whereas treatment group B (lamb stomach extract) had a very strong protective effect (P<0.00001).

The group (D) treated with the lowest dose of Vitamin B₁₂ had no protective effect. On the contrary, the protective effect achieved by the treatment group (E) was lower than that of the treatment group (B) in terms of the number of residual basal mucosal congestion observed in the treatment group B compared to the treatment group (E). The results indicated that the weight ratio of lamb stomach extract to Vitamin B₁₂ in the range of 100,000:4-100,000:1 produced a synergistic effect relative to lamb stomach extract or Vitamin B₁₂ alone. The combinations with lower ratios than 100,000:1 were advantageous in controlling residual basal mucosal congestion as compared with 100,000:4. By further reducing the weight ratio of Vitamin B₁₂ to 100000:0.2, the combination produced no synergistic effect. Lamb stomach extract Vitamin B₁₂ preparation composition was prepared according to the optimum combination B for subsequent studies.

### Research Method II:

1. Experimental animals: Forty-two healthy male Wistar rats, aged 4-6 weeks and weighing 140 to 180 g, were selected, and all the rats were kept in the experimental animal center at constant temperature and humidity (temperature 20-23° C., humidity about 50%), and were given 12 hours of light and 12 hours of dark environment circulation.
2. Experimental materials: N-methyl-N-nitro-N-nitroguanidine (MNNG) solution with a concentration of 1×105 g/L or sterile water was used as drinking water for rats. Sterile rat feed containing 0.03% ranitidine hydrochloride (hereinafter referred to as ranitidine feed) or normal standard sterile rat feed was used as rat feed. The contents of 50% ethanol solution or lamb stomach extract Vitamin B12 preparation were diluted in 0.9% sodium chloride solution or 0.9% sodium chloride solution for gastric perfusion.

Establishment of rat precancerous lesion model and drug intervention experiment: The rats in the dose group, the non-dose group and the negative control group were included in the drug intervention experiment, and the rats in the given group were given 0.2 g/kg Vitamin B12 once a day by gastric perfusion. Rats in the non-dose group and the negative control group were given 0.2 g/kg 0.9% sodium chloride solution once a day by gastric perfusion. The experiment lasted for 3 months.

Specimen collection and treatment: After the drug intervention experiment, the rats in the three groups were anesthetized and killed after fasting for 24-36 h without limiting drink. Blood samples, gastric juice and gastric tissue samples were collected. The gastric tissue samples were sent to the pathology department for dehydration, paraffin embedding, slicing and hematoxylin-eosin staining for pathological examination.

Observation and evaluation: Observe the general state (coat color, daily activities, food intake, water intake, weight changes), death, pH value of gastric juice, and pathological changes of gastric mucosa in each group (gross observation of mucosa, infiltration of inflammatory cells in mucosa, number of hyperplastic nodules on gastric mucosa surface, etc.). The pathological changes of gastric mucosa in rats were evaluated according to the Expert Consensus on Prevention, Control and Management of Helicobacter Pylori Infection in China Residents' Families (2021). The gastric precancerous lesion scores (1 to 3 points) of the rats in the dose group and the non-dose group were given. The rats without precancerous lesion (IN) were scored as 1 point, the rats with LGIN were scored as 2 points, and the rats with HGIN were scored as 3 points.

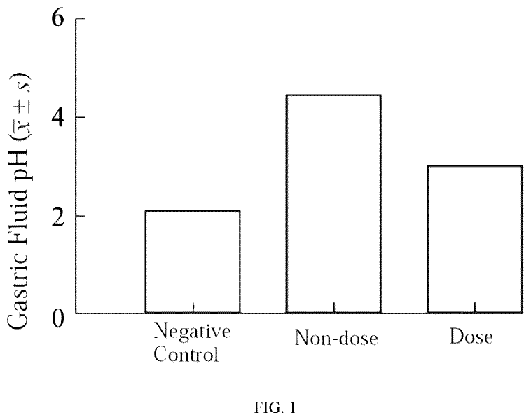

The comparison of the gastric juice pH values of the rats in the dose group, the non-dose group and the negative control group is shown in FIG. 1.

As can be seen from FIG. 1, the pH value of gastric juice in the dose group was higher than that in the negative control group (3.07±0.55 vs. 2.11±0.33) and lower than that in the non-dose group (3.07±0.55 vs. 4.45±0.72), and the differences were statistically significant (t=3.45, 2.80, all P<0.05). The gastric acid secretion of rats in the dose group was improved, which further reflected that the secretion function and even types of gastric mucosal epithelial cells might also be improved.

Pathological changes and precancerous lesion scores of gastric mucosa in rats were evaluated in dose group, non-dose group and negative control group.

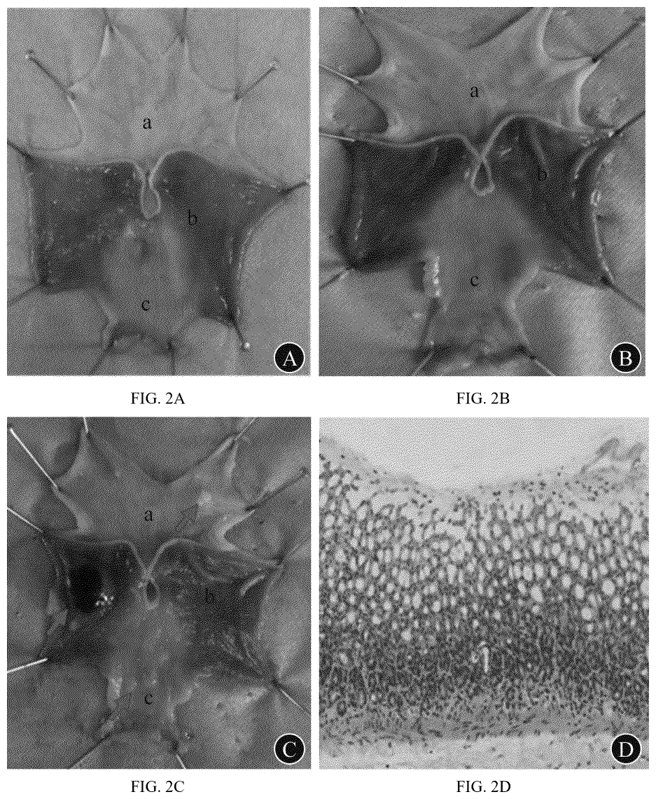

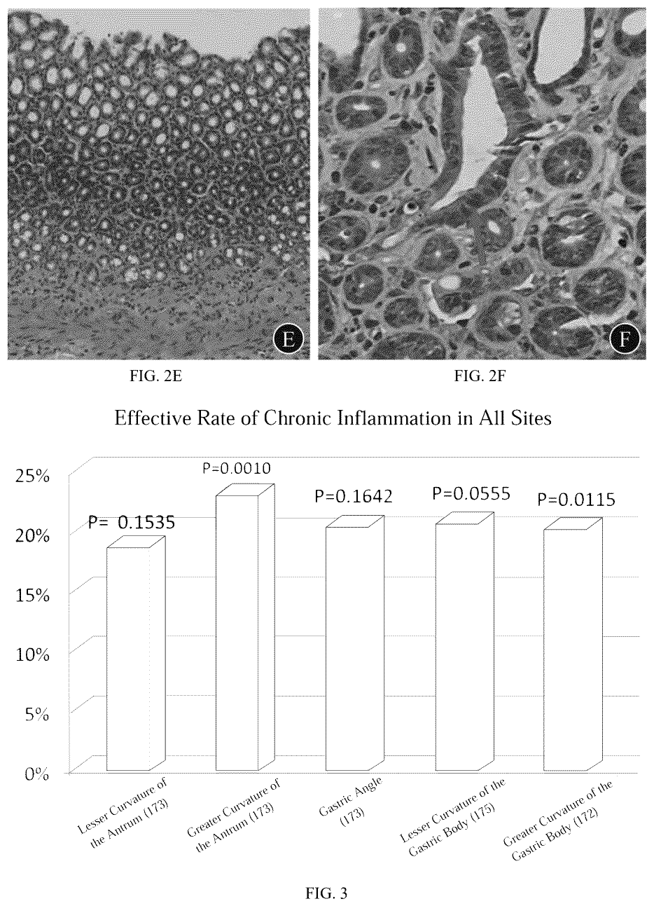

The gastric mucosa of rats in negative control group was dark orange with no abnormal growth on the surface (FIG. 2A). Except for a few white spots, there was no obvious abnormality in the gastric mucosa of the rats in the dose group, which was similar to the appearance and color of the gastric mucosa of the rats in the negative control group (FIG. 2B). In the non-dose group, tumor-like hyperplastic nodules with different sizes and numbers appeared on the gastric mucosa surface of the gastric fundus, the gastric body and the gastric antrum, and erosion and blood sinuses appeared in some cases (FIG. 2C). In the negative control group, the gland structure of gastric mucosa was normal, and there was basically no inflammatory cell infiltration or gland loss (FIG. 2D). In the dose group, there was a little inflammatory cell infiltration in the gastric mucosa, and the glands of lamina propria shrank, which was not worse than that at the end of modeling, and the density of glands of lamina propria in some individuals was richer than before (FIG. 2E). The gastric mucosal atrophy of rats in the non-dose group did not recover significantly or even worsened, and there were more atypical hyperplasia (FIG. 2F). These results indicate that the lamb stomach extract Vitamin B₁₂ preparation from can significantly reduce the risk of progression from precancerous state of gastric cancer to precancerous lesions in rats, suggesting that the lamb stomach extract Vitamin B₁₂ preparation may prevent precancerous lesions.

At the 6th week, the weight gain of rats in the dose group was greater than that in the non-dose group [(508.26±33.96) g vs (495.50±23.01) g], and the pH value of gastric juice was lower than that in the non-dose group (3.07±0.55 vs 4.45±0.72), with statistical significance (P<0.01). The number of hyperplastic nodules on the surface of gastric mucosa in each area of rats in the dose group was less than that in the non-dose group [6.00 (3.00, 7.00) vs. 11.00 (7.00, 13.00), 0.00 (0.00, 1.00) vs. 3.00 (2.00, 4.00). The score of gastric precancerous lesions in the dose group was lower than that in the non-dose group [(1.00±0.00) points vs (1.14±0.38) points], and the incidence of precancerous lesions was lower than that in the non-dose group (1/7 vs 5/7), and the differences were statistically significant (t=2.45, χ2=4.67, all P=0.031).

By establishing a rat model of chronic atrophic gastritis and intestinal metaplasia (research methods I and II), it is proved that the lamb stomach extract Vitamin B₁₂ preparation can promote the secretion of gastric acid and pepsin, promote the proliferation of main cells, parietal cells, G cells and D cells, regulate the secretion of neuroendocrine hormones, gastrin, somatostatin, prostaglandin E2, epidermal growth factor and its receptors, protect gastric mucosa and promote the regeneration of gastric mucosa glands, improve the bleeding volume and bleeding state of gastric mucosa, obviously improve and inhibit intestinal subtype, at the same time obviously increase the level of LPOSODJSH-PX in blood of rats, promote the growth of gastric mucosal gland cells, improve the synthesis of gastric mucosal cells, scavenge free radicals to protect gastric mucosa, treat and reverse chronic atrophic gastritis and intestinal metaplasia in rats, and have therapeutic effect on CAG.

### Example 5 Pathological Effects and Activities of Chymosin and Pepsin Activities in the Lamb Stomach Extract Vitamin B12 Preparation on the Treatment of Chronic Atrophic Gastritis with Intestinal Metaplasia

Trial: Treatment and Rational Dosage of Lamb Stomach Extract Vitamin B₁₂ Preparation in Patients with Moderate to Severe Atrophic Gastritis with Intestinal Metaplasia

BACKGROUND: Chronic atrophic gastritis with intestinal metaplasia is closely related to the development of gastric cancer. It was found that the bioactive enzymes contained in the lamb stomach extract Vitamin B₁₂ preparation may reverse gastric mucosal atrophy and intestinal metaplasia by promoting the proliferation of glands in the gastric mucosa, inhibiting apoptosis of glands, and suppressing oxidative stress in rats.

OBJECTIVE: To investigate the pathological effects of the bioactivity of lamb stomach extract Vitamin B₁₂ preparation in the treatment of chronic atrophic gastritis with intestinal metaplasia and its influencing factors.

METHODS: 173 patients with chronic atrophic gastritis with intestinal metaplasia who visited Xijing Hospital from June 2018 to June 2019 were included, and all patients were given 3-6 capsules of lamb stomach orally 3 times/d, using increasing the dose (or the number of times of taking), and gastroscopy and gastric mucosal tissue biopsy were carried out at 6 and 12 months of treatment, respectively. According to the changes of OLGA staging and OLGIM staging before and after treatment, the therapeutic effect was evaluated, and the factors affecting the therapeutic effect were discussed by univariate analysis and multivariate analysis.

RESULTS: After 6 months of treatment, the patients' OLGA staging efficiency was 49.7%, OLGIM staging efficiency was 32.9%, and the total efficiency was 64.7%. After 12 months of treatment, the OLGA staging efficiency was 56.4%, the OLGIM staging efficiency was 41.8%, and the total efficiency was 70.9%.

CONCLUSION: Lamb stomach significantly reduced OLGA and OLGIM stages in patients with chronic atrophic gastritis with intestinal metaplasia, suggesting that this drug may reduce the risk of gastric cancer in patients, and it has a better clinical application prospect in the prevention of gastric cancer.

### Objects and Methods

### I. Subject of the Study

One hundred and seventy-three patients with CAG with IM who visited the outpatient clinic of the Department of Gastroenterology at Xijing Hospital of Air Force Military Medical University from June 2018 to June 2019 were included and followed up. Inclusion criteria: (1) aged 18 to 70 years; (2) diagnosed with CAG with IM by upper gastrointestinal endoscopy and histopathological biopsy within the last three months. Exclusion criteria: (1) previously diagnosed with malignant tumors; (2) history of previous gastric surgeries; (3) in the period of breastfeeding or pregnancy; (4) suffering from endocrine diseases or autoimmune disease such as hypothyroidism, adrenal insufficiency, systemic lupus erythematosus, ankylosing spondylitis, systemic vasculitis; (5) suffering from serious mental illness; (6) refusing drug treatment; (7) patients diagnosed as gastric cancer or with high-grade intraepithelial neoplasia by upper gastrointestinal endoscopy and pathology; (8) patients suffering from serious hepatic and renal insufficiency; (9) patients with incomplete clinicopathological data. Data on patients' general condition, dietary habits, behavioral characteristics (smoking, drinking), disease history, and medication history were collected. There were 117 males and 56 females among the included cases; the average age was (54.4±8.8) years; the Hp infection rate was 79.8% (138/173).

### II. Research Methodology

1. Treatment Method: All patients were orally administered lamb stomach extract Vitamin B₁₂ preparation (capsule, 55 U/capsule) with 3-6 capsules 3 times/d for 12 months. At the same time, a quadruple regimen containing bismuth was given to Hp-infected patients and confirmed successful eradication. Gastroscopy and histopathologic examination of their mucosa were repeated at 6 and 12 months of treatment, respectively.
2. Gastric mucosal tissue biopsy: Biopsies were taken according to the new Sydney systematic histologic diagnostic criteria for chronic gastritis, and the five sites were the greater curvature side of the gastric sinus, the lesser curvature side of the gastric sinus, the greater curvature side of the gastric body, the lesser curvature side of the gastric body, and the gastric angle.
3. Assessment of efficacy: Calculate the patients' gastric mucosal atrophy and IM stage according to the OLGA and OLGIM gastric cancer risk staging assessment methods, and compare the staging changes before treatment as well as at 6 and 12 months after treatment. 1 or more stage reduction in OLGA and OLGIM staging was considered as effective treatment. Effectiveness in either stage was considered as overall effective, and effectiveness in both was considered as both effective.

### III. Statistical Analysis

SPSS 22.0 statistical software was applied. The one-sample Kolmogorov-Smirnov test was used to test the normality of continuous variables, non-normally distributed variables were expressed as M, normally distributed variables were expressed as x̅±s, and between-group comparisons were analyzed by analysis of variance (ANOVA); the count data were expressed as rate, and between-group comparisons were tested by χ² test, and multiple sample comparisons of hierarchical data were compared by the Kruskal-Wallis H test. The independent risk factors affecting the effectiveness of lamb stomach extract Vitamin B₁₂ preparation were analyzed by one-way and multifactorial regression analyses, and the odds ratios (ORs) and their 95% CIs were calculated. p<0.05 was regarded as statistically significant difference.

### Results

I. Changes in OLGA Staging and OLGIM Staging in Patients Treated with Lamb Stomach Extract Vitamin B₁₂ Preparation

173 patients with CAG with IM completed 6 months of treatment and were followed-up, and 110 completed 12 months of treatment and were followed-up. After 6 and 12 months of treatment, there was a significant increase in patients with stage 0 and stage I and a significant decrease in patients with stage II, III, and IV in OLGA and OLGIM staging (Tables 11 and 12).

TABLE 11
Distribution of OLGA and OLGIM staging before
and after 6 months of treatment (n)

| | | OLGA Staging | | | | | OLGIM Staging | | | | |
|---|---|---|---|---|---|---|---|---|---|---|---|
| Time | Cases | 0 | I | II | III | IV | 0 | I | II | III | IV |
| Pre-treatment | 173 | 4 | 12 | 68 | 56 | 33 | 63 | 34 | 41 | 29 | 6 |
| Post-treatment | 173 | 30 | 25 | 55 | 34 | 29 | 78 | 39 | 36 | 9 | 11 |

TABLE 12
Distribution of OLGA and OLGIM staging before
and after 6 months of treatment (n)

| | | OLGA Staging | | | | | OLGIM Staging | | | | |
|---|---|---|---|---|---|---|---|---|---|---|---|
| Time | Cases | 0 | I | II | III | IV | 0 | I | II | III | IV |
| Pre-treatment | 173 | 2 | 7 | 43 | 34 | 24 | 39 | 18 | 31 | 16 | 6 |
| Post-treatment | 173 | 28 | 17 | 26 | 21 | 18 | 48 | 39 | 12 | 9 | 10 |

Further analysis revealed that, after 6 months of treatment with lamb stomach extract Vitamin B₁₂ preparation, OLGA staging was reduced in 49.7% (86/173) of patients, OLGIM staging was reduced in 32.9% (57/173), OLGA and/or OLGIM staging was reduced in 64.7% (112/173), and OLGA and/or OLGIM staging was reduced in 17.9% (31/173) of patients had reduced OLGA and OLGIM staging (FIG. 1). After 12 months of treatment with lamb stomach extract Vitamin B₁₂ preparation, OLGA staging was reduced in 56.4% (62/110) of patients, OLGIM staging was reduced in 41.8% (46/110), OLGA and/or OLGIM staging was reduced in 70.9% (78/110), and OLGA and/or OLGIM staging was reduced in 27.3% (30/110) of patients had reduced OLGA and OLGIM staging (FIGS. 2A-2F). There was no statistically significant difference in the efficacy of lamb stomach extract Vitamin B₁₂ preparation compared between 6 months and 12 months treatment (P>0.05).

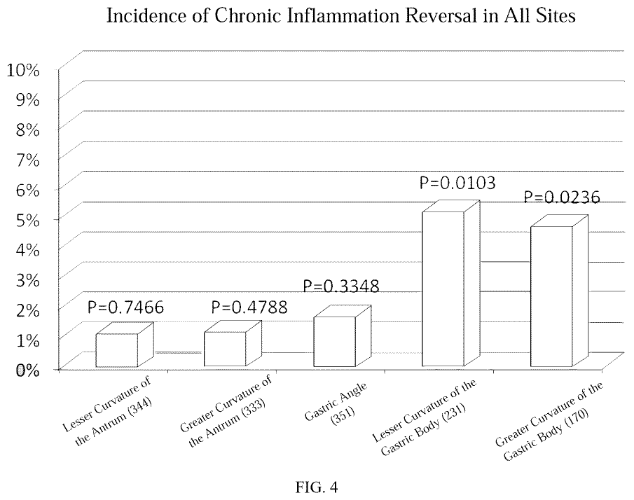

Before treatment, the proportion of high-risk staging (stage III and IV) in OLGA and OLGIM was higher. After treatment, the proportion of high-risk staging decreased significantly, while the proportion of low-risk staging increased significantly. The difference between the distribution of the staging after treatment compared with that before treatment was statistically significant (P<0.01; FIGS. 3 and 4). It indicated that the lamb stomach extract Vitamin B₁₂ preparation had a significant reversal effect on atrophy and IM. Further analysis showed that the percentage of OLGA high-risk staging was reduced more significantly than OLGIM staging after 6 and 12 months of treatment, and the difference was statistically significant (P<0.05). It suggested that the efficacy of lamb stomach extract Vitamin B₁₂ preparation in treating atrophy is superior to IM. In addition, the proportions of atrophy completely disappeared (OLGIM stage 0) and IM completely disappeared (OLGIM stage 0) were higher in 12 months of treatment than in 6 months of treatment, but the difference was not statistically significant (P>0.05).

II. The relationship between Hp infection and the efficacy of lamb stomach extract Vitamin B₁₂ preparation. The patients were divided into three groups according to the status of Hp infection, which were Hp negative group (patients who were always negative for Hp before enrollment), previous Hp eradication group (patients who had been successfully eradicated from Hp three months before and tested negative before enrollment), and pre-dose Hp eradication group (patients who were positive for Hp at the time of enrollment, and were successfully eradicated from Hp before taking the lamb stomach extract Vitamin B₁₂ preparation). The difference in OLGA and OLGIM staging efficiency at 6 months of treatment was not statistically significant in all three groups (P>0.05). At 12 months of treatment, the difference in OLGA staging and OLGIM staging efficiency was not statistically significant in each group (P>0.05). Although the use of lamb stomach extract Vitamin B₁₂ preparation after the eradication of Hp had no significant effect on the effective rate of OLGA staging, it could more effectively reduce the OLGIM staging of patients (P<0.05; Table 13).

TABLE 13
Analysis of OLGA and OLGIM efficacy rates after treatment
in patients with different Hp status n (%)

| | 6 months treatment | | | 12 months treatment | | |
|---|---|---|---|---|---|---|
| | | OLGA | OLGIM | | OLGA | OLGIM |
| Groups | Cases | staging | staging | Cases | staging | staging |
| Hp negative group | 35 | 18(51.4) | 14(40.0) | 26 | 15(57.7) | 7(26.9) |
| Previous Hp | 112 | 55(49.1) | 36(32.1) | 61 | 34(55.7) | 25(41.0)* |
| eradication group | | | | | | |
| Pre-dose Hp | 26 | 13(50.0) | 7(26.9) | 23 | 13(56.5) | 14(60.9)*^{#} |
| eradication group | | | | | | |

### Example 6 Phase IV Clinical Study of a Lamb Stomach Extract Vitamin B₁₂ Preparation Composition for the Treatment of Chronic Atrophic Gastritis

To evaluate the efficacy and safety of lamb stomach extract Vitamin B₁₂ preparation for the treatment of chronic atrophic gastritis, the effect of lamb stomach extract Vitamin B₁₂ preparation on the reversal of glandular atrophy in chronic atrophic gastritis was investigated, and pharmacoeconomics data were collected during the course of treatment.

### Research Methodology

A randomized, double-blind, multicenter clinical study was conducted using placebo as the control and histopathological changes as the primary indicator of efficacy. Successfully screened subjects was randomized in a 2:1 ratio to the trial and placebo groups.

### Evaluation Criteria

### Efficacy Indicators.

(1) Effective rate: Effectiveness was defined as a ≥1-point reduction in histopathology score from baseline. Histopathologic evaluation was performed by taking biopsies from 5 sites, including chronic inflammation, active, atrophy, and intestinal epithelial metaplasia, in the lesser curvature of the gastric antrum, greater curvature of the gastric antrum, angle of the stomach, lesser curvature of the gastric body, and greater curvature of the gastric body, respectively, which were categorized into 4 grades: normal (0 points), mild (1 point), moderate (2 points), and severe (3 points).

The effective rate was calculated and evaluated at five sites, namely, the lesser curvature of the antrum, the greater curvature of the antrum, the angle of the stomach, the lesser curvature of the gastric body and the greater curvature of the gastric body, respectively. Cases whose histopathologic evaluations were normal at the time of enrollment were not included in the calculation of the effective rate. The comprehensive evaluation of the effective rate was carried out by the following two methods:
(i) Comprehensive evaluation 1: The scores of the most severe sites before and after treatment were selected separately to calculate the effective rate;
(ii) Comprehensive evaluation 2: The sites with the most severe chronic inflammation, activity, atrophy, and intestinal epithelial metaplasia in the subjects at the time of enrollment were selected as the target observation sites, respectively. The effective rate of the target observation sites after 180 days of treatment was evaluated. When multiple sites had equal scores at enrollment, the one with the smallest change in score before and after treatment was selected for evaluation.

In this case, the comprehensive evaluation 1 methodology was used as the main indicator for the evaluation of effective rate.
(2) Incidence of Reversal: The incidence of reversal was defined as a reduction of ≥2 points from baseline in the 180-day histopathology score of enrolled moderate and severe patients.

The incidence of reversal was calculated and evaluated at five sites: the lesser curvature of the antrum, the greater curvature of the antrum, the gastric angle, the lesser curvature of the gastric body and the greater curvature of the gastric body, and the cases with normal and mild histopathologic evaluations at the time of enrollment were not included in the calculation of the incidence of reversal. The comprehensive evaluation of the incidence of reversal was performed by the following two methods:
(i) Comprehensive evaluation 1: The incidence of reversal was calculated by selecting the scores of the most severe sites before and after treatment, respectively;
(ii) Comprehensive evaluation 2: The sites with the most severe chronic inflammation, activity, atrophy and intestinal epithelial metaplasia in the subjects at the time of enrollment were selected as the target observation sites, respectively, and the incidence of reversal of the target observation sites after 180 days of treatment was evaluated. When multiple sites had equal scores at enrollment, the one with the smallest change in score before and after treatment was selected for evaluation.

In this case, the comprehensive evaluation 1 methodology was used as the main indicator for the evaluation of incidence of reversal.
(3) Histopathology score: The histopathology score evaluated the sum of the histopathology scores of the five sites of the lesser curvature of the antrum, greater curvature of the antrum, angle of the stomach, lesser curvature of the gastric body, and greater curvature of the gastric body, the most severe pathology score of each site, and the histopathology scores of each site, respectively. The evaluation of histopathologic evaluation included chronic inflammation, activity, atrophy, and intestinal epithelial metaplasia.
(4) Gastroscopic gastric mucosal changes: These included endoscopic manifestations of atrophic gastritis (red and white mucosa, predominantly white mucosa, flattened folds, disappearing folds, vascularization, and granular or nodular mucosa) and concomitant endoscopic vesiculation or bile reflux (flattened vesicles, elevated vesiculation, hemorrhagic punctate spots, mucosal congestion, and bile reflux). The cardia, gastric body, gastric angle, gastric sinus, gastric fundus and pylorus were evaluated separately.
(5) Clinical symptom score: 7 items including abdominal discomfort, loss of appetite, abdominal distension, dyspeptic diarrhea, abdominal pressure, acid reflux and nausea and vomiting. Among them, the total abdominal pain score included 3 items of abdominal discomfort, abdominal distension and abdominal pressure, and the total score outside abdominal pain included 4 items of loss of appetite, dyspeptic diarrhea, acid reflux and nausea and vomiting.

### Security Indicators

Adverse events;
Laboratory tests: routine blood (erythrocytes, hemoglobin, leukocytes, platelets, and neutrophils), blood biochemistry (ALT, AST, BUN, Cr, and fasting glucose), routine urine (urinary leukocytes, urinary erythrocytes, and urinary glucose), and routine stool and occult blood tests;
Electrocardiogram;

General examination: including temperature, pulse, blood pressure, and respiration.

Statistical Methods: All statistical tests were performed using a two-sided test, and a p-value less than or equal to 0.05 would be considered statistically significant for the differences tested. (Unless otherwise specified).

Measurement information is statistically mentioned by mean, median, standard deviation, maximum, minimum, 25% and 75% quartiles; count or rank information is expressed in terms of frequency and frequency.

### Analysis of the Efficacy

### 1. Effective Rate of Chronic Inflammation in all Sites

Lamb stomach extract Vitamin B₁₂ preparation was more effective than the placebo group in chronic inflammation of the greater curvature of the sinus and the greater curvature of the body of the stomach in patients with chronic atrophic gastritis (P<0.05). The results are shown in FIG. 3.

### 2. Incidence of Reversal of Chronic Inflammation in all Sites

The incidence of reversal of chronic inflammation in the greater curvature of the gastric body and lesser curvature of the gastric body in patients with chronic atrophic gastritis was higher in the lamb stomach extract Vitamin B₁₂ preparation than in the placebo group (P<0.05). The results are shown in FIG. 4.

### 3. Effective Rate of Atrophy in all Sites

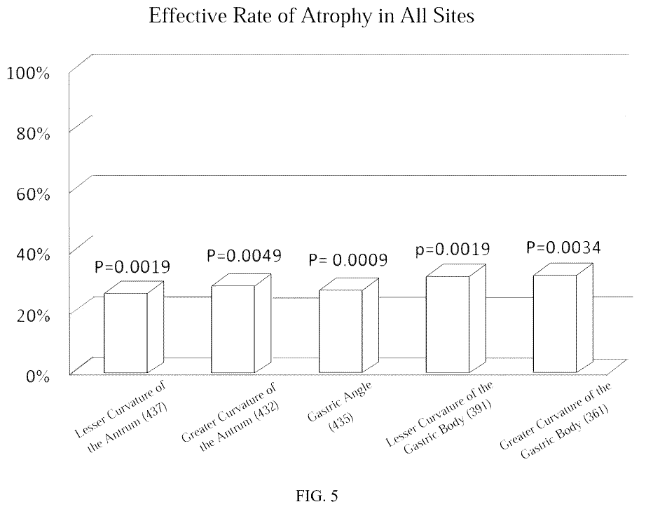

Lamb stomach extract Vitamin B₁₂ preparation was more effective than the placebo group in atrophy of the greater curvature of the gastric body, lesser curvature of the gastric body, gastric horns, lesser curvature of the gastric sinus and greater curvature of the gastric sinus in patients with chronic atrophic gastritis (P<0.05). The results are shown in FIG. 5.

### 4. Incidence of Reversal of Atrophy in all Sites

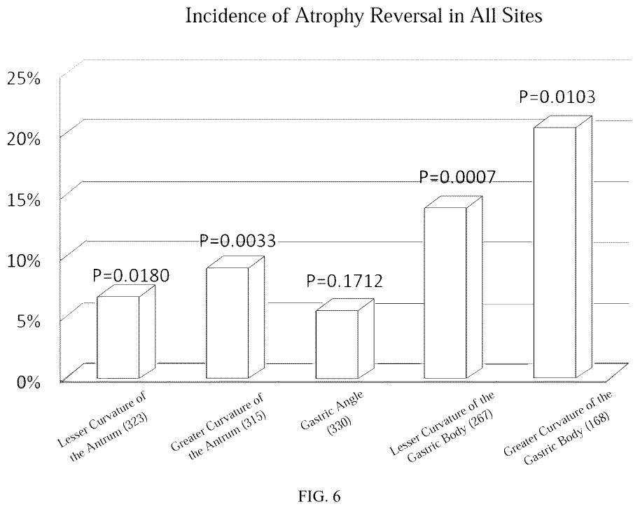

Lamb stomach extract Vitamin B₁₂ preparation had a higher incidence of reversal of atrophy in the greater curvature of the gastric body, lesser curvature of the gastric body, lesser curvature of the gastric sinus and greater curvature of the gastric sinus in patients with chronic atrophic gastritis than that in the placebo group (P<0.05). The results are shown in FIG. 6.

### 5. Effective Rate of Intestinal Epithelial Metaplasia in all Sites

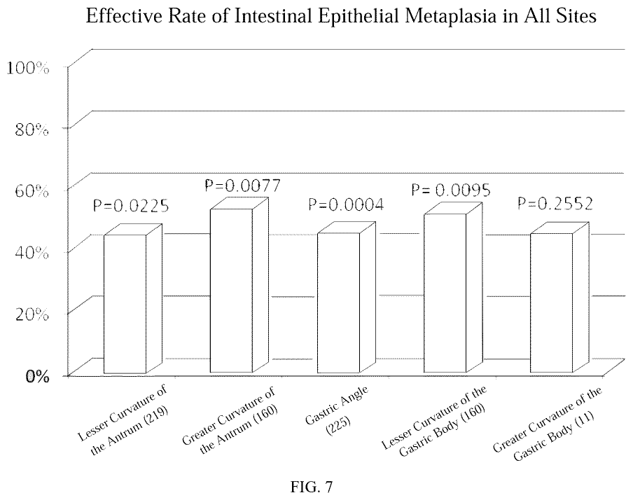

Lamb stomach extract Vitamin B₁₂ preparation was more effective than the placebo group against intestinal epithelial metaplasia in the lesser curvature of the gastric body, gastric horns, lesser curvature of the gastric sinus, and greater curvature of the gastric sinus in patients with chronic atrophic gastritis (P<0.05). The results are shown in FIG. 7.

### 6. Incidence of Reversal of Intestinal Epithelial Metaplasia by Site

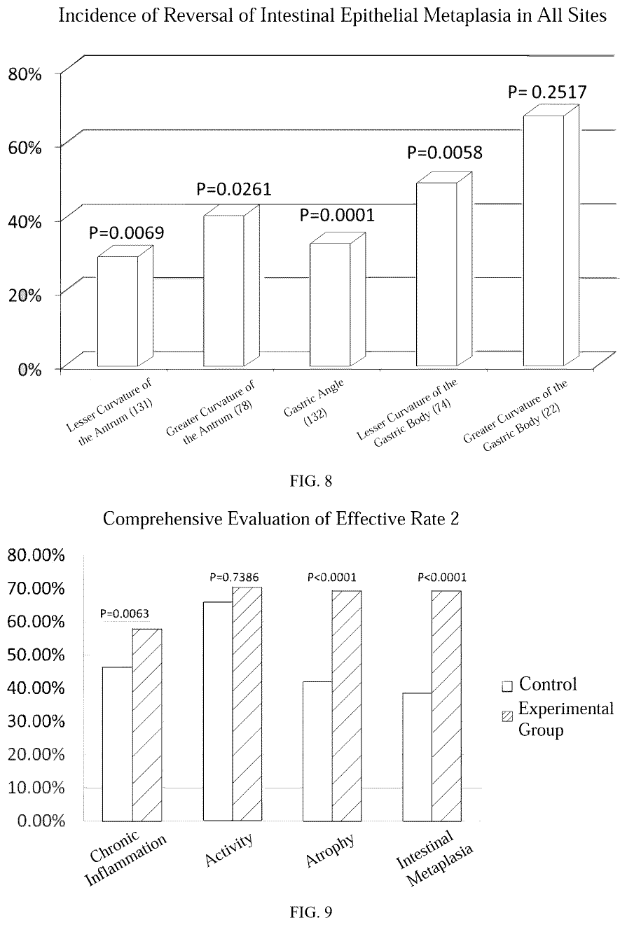

Lamb stomach extract Vitamin B₁₂ preparation showed a higher incidence of reversal of intestinal epithelial metaplasia in the lesser curvature of the gastric body, gastric horns, lesser curvature of the gastric sinus and greater curvature of the gastric sinus in patients with chronic atrophic gastritis than that in the placebo group (P<0.05). The results are shown in FIG. 8.

### 7. Efficient Integrated Evaluations

Lamb stomach extract Vitamin B₁₂ preparation was more effective than the placebo group in chronic inflammation, atrophy and intestinal metaplasia of the stomach in patients with chronic atrophic gastritis (P<0.05). The results are shown in FIG. 9.

### 8. Comprehensive Evaluation of the Incidence of Reversals

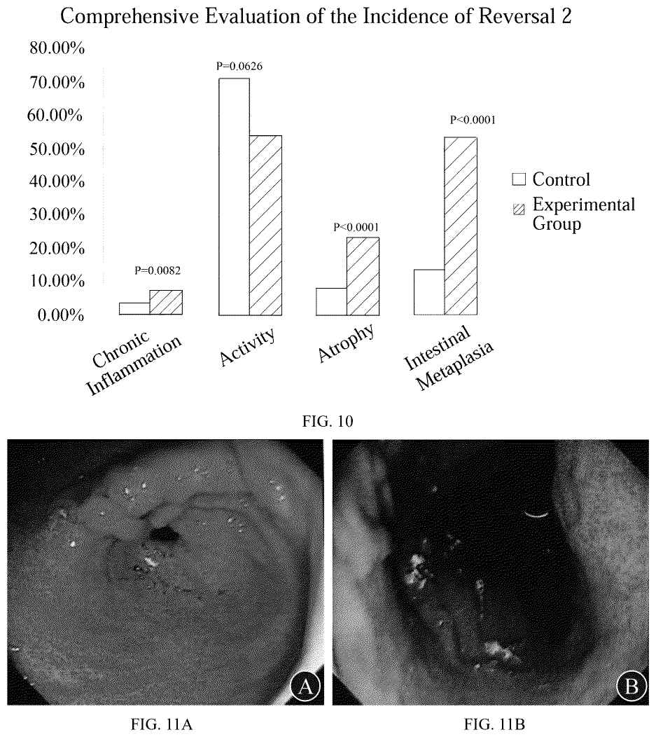

Lamb stomach extract Vitamin B₁₂ preparation had a higher incidence of gastric chronic inflammation, atrophy and intestinal metaplasia reversal in patients with chronic atrophic gastritis than in the placebo group (P<0.05). The results are shown in FIG. 10.

### Effective Results:

### Effective Rate

Comprehensive evaluation 1 was used. That is, the most severe histopathologic evaluation before and after treatment was taken to calculate the effective rate: Comparison of chronic inflammation efficiency between groups: efficiency FAS (PPS), placebo 10.60% (12.13%), Lamb stomach 15.70% (17.86%), P=0.0411 for FAS result and P=0.0492 for PPS result by two-way unordered CMH-χ² test, and the differences between the groups were statistically significant.

Comparison of activity efficiency between groups: efficiency FAS (PPS), placebo 34.48% (38.16%), lamb stomach 41.07% (45.58%), P=0.5336 for FAS result and P=0.4601 for PPS result by two-way unordered CMH-χ² test, and none of the differences between groups were statistically significant.

Comparison of atrophy efficiency between groups: efficiency FAS (PPS), placebo 7.80% (8.79%), lamb stomach 16.01% (18.60%), P=0.0010 for FAS results and P=0.0008 for PPS results by two-way unordered CMH-χ² test, and the differences between the groups were statistically significant.

The FAS result was P=0.0003 and the PPS result was P=0.0003 by the two-way unordered CMH-χ² test, and the differences between the groups were statistically significant. Since there was a baseline imbalance in the most severe intestinal epithelial metaplasia, a logistic regression model was fitted with the most severe intestinal epithelial metaplasia at baseline as a covariate and the intestinal epithelial metaplasia efficiency as a response variable to further examine the intestinal epithelial metaplasia efficiency. The results showed a P=0.0001 for the FAS result and a P=0.0001 for the PPS result, and the difference between groups remained statistically significant.

Comprehensive evaluation 2 was used. That is, the site with the heaviest pre-treatment histopathologic evaluation was taken as the target site for calculating the effective rate:

Comparison of chronic inflammation efficiency between groups: efficiency FAS (PPS), 39.93% (46.44%) for placebo and 49.21% (57.56%) for Lamb stomach, P=0.0103 for FAS result and P=0.0063 for PPS result by two-way unordered CMH-χ² test, and the differences between the groups were statistically significant.

Comparison of active efficiency between groups: efficiency FAS (PPS), placebo 58.62% (65.79%), lamb stomach 63.69% (70.07%), P=0.6068 for FAS result and P=0.7386 for PPS result by two-way unordered CMH-χ² test, and none of the differences between groups were statistically significant.

Comparison of atrophy efficiency between groups: efficiency FAS (PPS), 36.17% (41.84%) for placebo and 59.61% (69.13%) for lamb stomach by two-way unordered CMH-χ² test, P<0.0001 for FAS result and P<0.0001 for PPS result, and the difference between the groups were all statistically significant.

Comparison of the efficiency of intestinal epithelial metaplasia between groups: efficiency FAS (PPS), placebo 34.29% (38.31%), lamb stomach 62.24% (69.25%), by two-way unordered CMH-χ² test, the result of FAS was P<0.0001, and the result of PPS was P<0.0001, and the difference between the groups were all statistically significant.

### Incidence of Reversal

Comprehensive evaluation 1 was used. That is, the incidence of reversal was calculated by taking the heaviest histopathologic evaluation before and after treatment:

Comparison of the incidence of chronic inflammation reversal between groups: no reversal occurred in either the lamb stomach or placebo groups.

Comparison of the incidence of activity reversal between groups: incidence of reversal FAS (PPS), placebo 33.33% (35.48%) and lamb stomach 20.73% (22.54%), P=0.1044 for FAS results and P=0.3446 for PPS results by two-way unordered CMH-χ² test; And none of the differences between groups were statistically significant.

Comparison of the incidence of atrophy reversal between groups: incidence of reversal FAS (PPS), placebo 0.00% (0.00%), lamb stomach 1.13% (1.34%), P=0.0818 for FAS results and P=0.0810 for PPS results by two-way unordered CMH-χ² test; and none of the differences between groups were statistically significant.

Comparison of the incidence of reversal of intestinal epithelial metaplasia between groups: the incidence of reversal FAS (PPS), placebo 3.64% (4.08%), lamb stomach 13.62% (14.67%) was statistically significant by the two-way unordered CMH-χ² test, with the FAS result of P=0.0079 and the PPS result of P=0.0139, and the differences were statistically significant between groups.

Since there was a baseline imbalance in the most severe intestinal epithelial metaplasia, a logistic regression model was fitted with the most severe intestinal epithelial metaplasia at baseline as a covariate and the incidence of intestinal epithelial metaplasia reversal as a dependent variable to further examine the incidence of intestinal epithelial metaplasia reversal. The results showed that the FAS result was P=0.0083 and the PPS result was P=0.0104, and the difference between groups remained statistically significant.

Comprehensive evaluation 2 was used. That is, the site with the heaviest pre-treatment histopathologic evaluation was taken as the target site to calculate the incidence of reversal:

Comparison of the incidence of chronic inflammation reversal between groups: incidence of reversal FAS (PPS), placebo 1.59% (1.87%), lamb stomach 6.10% (6.68%), P=0.0057 for FAS results and P=0.0082 for PPS results by two-way unordered CMH-χ² test, and differences between groups were all statistically significant.

Comparison of the incidence of activity reversal between groups: incidence of reversal FAS (PPS), placebo 63.89% (70.97%), lamb stomach 50.00% (53.52%), statistically significant difference between groups by the two-way unordered CMH-χ² test for the FAS result of P=0.0424, and for the PPS result of P=0.0626; and none of the differences between groups were statistically significant.

Comparison of the incidence of atrophy reversal between groups: the incidence of reversal rate FAS (PPS), placebo 6.39%(7.14%), lamb stomach 19.96%(23.16%), the result of FAS was P<0.0001 and the result of PPS was P<0.0001 by two-way unordered CMH-χ² test, and the difference between the groups was statistically significant.

Comparison of the incidence of reversal of intestinal epithelial metaplasia between groups: the incidence of reversal FAS (PPS), placebo 11.82% (13.27%), lamb stomach 47.86% (53.33%), by two-way unordered CMH-χ² test, FAS result was P<0.0001, PPS result was P<0.0001, and the differences between groups were all statistically significant.

### Histopathology Score

Comparison of the sum of histopathological scores of five sites of chronic inflammation between groups: pre-treatment mean SD, placebo FAS (PPS) 8.32±2.35 (8.33±2.33), lamb stomach FAS (PPS) 8.19±2.28 (8.21±2.22); post-treatment mean±SD, placebo FAS (PPS) 8.63±2.28 (8.72±2.25), lamb stomach FAS (PPS) 8.19±2.14 (8.22±2.06); (post-treatment−pre-treatment) difference mean±SD, placebo FAS (PPS) 0.31±1.72 (0.38±1.81), lamb stomach FAS (PPS) −0.00±2.04 (0.00±2.20), FAS by paired group t test result was P=0.0246 and PPS result was P=0.0203, and all differences between groups were statistically significant.

Comparison of the sum of histopathologic scores of the five sites of activity between groups: pre-treatment mean±SD, placebo FAS (PPS) 0.95±1.96 (0.96±1.90), lamb stomach FAS (PPS) 1.01±2.03 (1.02±1.98); post-treatment mean±SD, placebo FAS (PPS) 1.21±2.32 (1.28±2.38), lamb stomach FAS (PPS) 1.27±2.35 (1.34±2.37); (post-treatment−pre-treatment) difference mean±SD, placebo FAS (PPS) 0.26±2.11 (0.32±2.20), lamb stomach FAS (PPS) 0.26±2.13 (0.32±2.28) by paired group t-test, FAS result was P=0.9909 and PPS result was P=0.9997, none of the differences between groups were statistically significant.

Comparison of the sum of histopathologic scores of the five sites of atrophy between groups: pre-treatment mean±SD, placebo FAS (PPS) 7.77±3.05 (7.75±2.98), lamb stomach FAS (PPS) 8.21±3.00 (8.25±3.00); post-treatment mean±SD, placebo FAS (PPS) 9.17±2.86 (9.38±2.66), lamb stomach FAS (PPS) 8.75±2.56 (8.89±2.45); (post-treatment−pre-treatment) difference mean±SD, placebo FAS (PPS) 1.40±2.53 (1.63±2.59), lamb stomach FAS (PPS) 0.54±2.92 (0.63±3.13) by paired t-test, FAS result was P<0.0001 and PPS result was P<0.0001, and all differences between groups were statistically significant.

Comparison of the sum of histopathologic scores of five sites of intestinal epithelial metaplasia between groups: pre-treatment mean±SD, placebo FAS (PPS) 2.31±2.75 (2.45±2.79), lamb stomach FAS (PPS) 2.90±3.18 (3.01±3.20); post-treatment mean±SD, placebo FAS (PPS) 2.97±3.42 (3.25±3.52), lamb stomach FAS (PPS) 2.78±3.07 (2.89±3.09); (post-treatment−pre-treatment) difference mean±SD, placebo FAS (PPS) 0.66±1.90 (0.80±2.02), lamb stomach FAS (PPS) −0.12±2.19 (−0.13±2.36), as determined by the paired t-test. The results of FAS were P<0.0001 and PPS were P<0.0001, and the differences between groups were statistically significant.

### Clinical Symptom Score

Changes in total clinical symptom scores at the end of treatment were analyzed by analysis of covariance, with no statistically significant differences between subgroups (FAS: P=0.4637, PPS: P=0.9235) and a consistent trend of differences between groups between centers (FAS: P=0.8169, PPS: P=0.9044);

The mean (95% confidence interval) of the difference between placebo and lamb stomach correction was 0.24 (−0.40,0.88) for the FAS results and 0.03 (−0.67,0.74) for the PPS results, with no statistically significant differences between groups.

The corrected mean (95% confidence interval) FAS result for the change in total clinical symptom score at the end of placebo treatment was −0.41 (−1.22,0.40), and the PPS result was −0.37 (−1.35,0.61), with neither difference statistically significant. The corrected mean (95% confidence interval) FAS result for the change in total clinical symptom score at the end of lamb stomach treatment was −0.65 (−1.37,0.07), and the PPS result was −0.41 (−1.32,0.51), and none of the differences were statistically significant.

Changes in total abdominal pain scores at the end of treatment were analyzed by analysis of covariance, with no statistically significant differences between subgroups (FAS: P=0.0976, PPS: P=0.5786) and a consistent trend of differences between groups between centers (FAS: P=0.9651, PPS: P=0.9743);

The mean (95% confidence interval) of the difference between placebo and lamb stomach corrections was 0.38 (−0.07,0.82) for the FAS results and 0.14 (−0.35,0.63) for the PPS results, with no statistically significant differences between groups.

The corrected mean (95% confidence interval) FAS result for the change in total abdominal pain score at the end of placebo treatment was 0.07 (−0.50,0.63), and the PPS result was −0.14 (−0.81,0.54), with neither difference statistically significant. The corrected mean (95% confidence interval) FAS result for the change in total abdominal pain score at the end of lamb stomach treatment was −0.31 (−0.81,0.20), and the PPS result was −0.27 (−0.91,0.36), and none of the differences were statistically significant.

Changes in total scores of clinical symptoms other than abdominal pain at the end of treatment were analyzed by analysis of covariance, with no statistically significant differences between subgroups (FAS: P=0.7218, PPS: P=0.6078), and a consistent trend of differences between groups between centers (FAS: P=0.4674, PPS: P=0.7934);

The mean (95% confidence interval) of the difference between placebo and lamb stomach correction was −0.07 (−0.46,0.32) for the FAS results and −0.11 (−0.53,0.31) for the PPS results, with no statistically significant differences between groups.

The corrected mean (95% confidence interval) FAS result for the change in total clinical symptom scores other than abdominal pain score at the end of placebo treatment was −0.40 (−0.89,0.09), and the PPS result was −0.21 (−0.80,0.38), with neither difference statistically significant. The corrected mean (95% confidence interval) FAS result for the change total clinical symptom scores other than abdominal pain score at the end of lamb stomach treatment was −0.33 (−0.77,0.11), and the PPS result was −0.10 (−0.65,0.45), and none of the differences were statistically significant.

As can be seen from the above results:
1. Lamb stomach extract Vitamin B₁₂ preparation is generally effective in chronic atrophic gastritis, with better histologic improvement.
2. It is effective in chronic inflammation, atrophy, and histology of intestinal metaplasia, especially better for intestinal metaplasia, and effective in reversing intestinal epithelial metaplasia.

### Example 7 Big Data Real-World Study: Reversal of Chronic Atrophic Gastritis and Intestinal Metaplasia by Lamb Stomach Extract Vitamin B₁₂ Preparation Composition

Patients with CAG treated with lamb stomach extract Vitamin B₁₂ preparation were analyzed before and after their own control.
1. Treatment: Lamb stomach extract Vitamin B₁₂ preparation (capsule, 55 U/capsule). After six months of regular treatment (110 U/times, 3 times/d), upper gastrointestinal endoscopy and biopsy of the gastric mucosa were performed to evaluate the treatment effect, and urea breath test was performed to detect the presence of Helicobacter pylori infection. Treatment was initiated with eradication of H. pylori infection, if any, followed by initiation of lamb stomach extract Vitamin B₁₂ preparation after 2 weeks.
2. Endoscopic biopsy: 5 biopsy specimens were taken according to the requirements of the new Sydney standard. That is, 2-3 cm from the pylorus on the lesser curvature side of the gastric sinus, 2-3 cm from the pylorus on the greater curvature side of the gastric sinus, 4 cm from the angle of the stomach on the lesser curvature side of the gastric body, 8 cm from the cardia on the greater curvature side of the gastric body, and 5 sites at the angle of the stomach.
3. Determination of gastric mucosal inflammatory status and atrophy condition: the inflammatory status of gastric mucosa was assessed using the OLGA grading system for chronic gastritis, and was divided into grades 0 to IV on the basis of the scoring level and was recorded as 0 to 4 points, of which 1 to 2 points were defined as mild inflammation, and 3 to 4 points were defined as severe inflammation; atrophy and intestinal metaplasia were staged by using the OLGA and OLGIM staging systems for chronic gastritis, respectively, and the combined scoring results were categorized into stages 0 to IV, with stages 0 to II defined as low staging and stages III or IV as high staging.
4. Efficacy indexes: OLGA or OLGIM stage decreased by ≥1 stage after treatment compared with that before treatment as effective reversal of atrophy or enterocolitis; OLGA or OLGIM stage increased by ≥1 stage after treatment compared with that before treatment as progression of atrophy or enterocolitis. Effective reversal rate (%)=number of cases with effective reversal after treatment/total number of cases×100%; progression rate (%)=number of cases with progression after treatment/total number of cases×100%; effective rate of total reversal (%)=number of cases with effective reversal of atrophy or intestinal metaplasia after treatment/total number of cases×100%.

### Statistical Methods

SPSS 25.0 software was applied for statistical analysis. Measurement information with normal distribution was expressed as x̅±s and independent sample t-test was used for comparison between groups. Count data were expressed as cases and percentages, and the chi-square test was used for intergroup comparisons; the Wilcoxon rank-sum test was used for intergroup comparisons of rank data. Multifactorial logistic regression was used to analyze the influencing factors of drug treatment effect, and the ratio and 95% confidence interval (95% CI) were calculated. p<0.05 was regarded as statistically significant difference.

### General Information and Clinical Characteristics

Of the 240 patients, 146 were male and 94 were female; the mean age was (53.8±8.0) years; BMI <18.5 kg/m² 17 cases (7.1%), BMI 18.5˜<24.0 kg/m² 147 cases (61.2%), BMI 24.0-28.0 kg/m² 66 cases (27.5%), BMI >28.0 kg/m² 10 cases (4.2%); blood type A 44 cases (18.3%), blood type B 47 cases (19.6%), blood type O 52 cases (21.7%), blood type AB 22 cases (9.2%), and blood type unknown 75 cases (31.2%); people with high school education or above 155 cases (64.6%); urban residents 165 cases (68.8%); with a family history of gastric cancer 56 cases (23.3%); history of cholecystectomy 14 cases (5.8%); hypertension 43 cases (17.9%); diabetes mellitus 14 cases (5.8%); Helicobacter pylori positive 76 cases (31.7%); gastric mucous membrane inflammation scores from low to high were 1 point 4 cases (1.7%), 2 points 158 cases (65.8%), 3 points 65 cases (27.1%), 4 points 13 cases (5.4%).

### Analysis of Treatment Effects

As shown in Table 14, after six months of treatment with lamb stomach extract Vitamin B₁₂ preparation, the proportion of patients with OLGA and OLGIM low stage (0-II) was higher than that before the treatment [62.9% (151/240) versus 50.8% (122/240) and 80.8% (194/240) versus 72.5% (174/240)], and the difference was statistically significant in all cases (χ²=7.143, 4.658, all P<0.05). As shown in Table 15, the proportion of patients with severe inflammation of the gastric mucosa (inflammation score of 3 to 4) was lower after treatment than before treatment [23.3% (56/240) versus 32.5% (78/240)], and the difference was statistically significant (χ²=5.011, P<0.05). The proportion of patients with H. pylori positivity after treatment was lower than that before treatment [10.8% (26/240) versus 31.7% (76/240)], and the difference was statistically significant (χ²=31.124, P<0.05). Of the 10 patients with low-grade intraepithelial neoplasia on initial treatment, 8 had undetectable allopatric metaplasia on post-treatment review. No adverse reactions occurred in any of the 240 patients during treatment.

TABLE 14
Distribution of OLGA, OLGIM staging before and after treatment
with lamb stomach extract Vitamin B₁₂ preparation (cases)

| | | OLGA Staging | | | | | OLGIM Staging | | | | |
|---|---|---|---|---|---|---|---|---|---|---|---|
| Time | Cases | 0 | I | II | III | IV | 0 | I | II | III | IV |
| Pre-treatment | 240 | 10 | 13 | 99 | 84 | 34 | 24 | 61 | 89 | 47 | 19 |
| Post-treatment | 240 | 35 | 20 | 96 | 63 | 26 | 28 | 89 | 77 | 32 | 14 |

Note:
OLGA is the gastritis evaluation system; OLGIM is the gastritis evaluation system based on intestinal metaplasia. Stage 0-II is low OLGA or OLGIM staging; stage III-IV is high OLGA or OLGIM staging.

TABLE 15
Inflammatory status of gastric mucosa and Helicobacter pylori
infection in patients before and after treatment with lamb stomach
extract Vitamin B₁₂ preparation (cases)

| | | Inflammation score | | | | H. pylori | |
|---|---|---|---|---|---|---|---|
| Time | Cases | 1 Point | 2 Point | 3 Point | 4 Point | Positive | Negative |
| Pre-treatment | 240 | 4 | 158 | 65 | 13 | 76 | 164 |
| Post-treatment | 240 | 4 | 180 | 51 | 5 | 26 | 214 |

After six months of treatment with lamb stomach extract Vitamin B₁₂ preparation, the reversal rates of gastric mucosal atrophy and intestinal metaplasia in patients were 45.4% (109/240) and 37.9% (91/240), respectively, and the total reversal rate was 62.9% (151/240), of which 35 cases of atrophy completely regressed, 15 cases of intestinal metaplasia completely regressed, and 8 cases of atrophy and intestinal metaplasia were both completely regressed, respectively. 21.3% (51/240) and 21.3% (51/240) of patients had progression of atrophy and intestinal metaplasia, respectively; atrophy and intestinal metaplasia staging remained unchanged in 33.3% (80/240) and 40.8% (98/240) of patients. After treatment, 53.4% (63/118) and 54.5% (36/66) of patients with high OLGA and OLGIM staging, respectively, reversed to low staging, and 27.0% (33/122) and 15.5% (16/174) of patients with low OLGA and OLGIM staging, respectively, progressed to high staging. The difference in the composition ratio of patients with high and low OLGA and OLGIM staging before and after treatment was statistically significant (χ²=19.492, 10.013, both P<0.05). The proportion of patients with gastric mucosal atrophy in the OLGA staging I˜IV who had the reversal of gastric mucosal atrophy was 4/13, 30.3% (30/99) 59.5% (50/84), and 70.6% (24/34), respectively. The proportions of reversal of intestinal metaplasia in OLGIM stages I to IV were 11.5% (7/61), 49.4% (44/89) 51.1% (24/47), and 13/19, respectively. Except for the statistically insignificant difference in the rate of reversal of intestinal metaplasia in patients with OLGIM stage II compared with those with OLGIM stage III (P>0.05), the rate of reversal in patients with high OLGA and OLGIM stages was higher than that in those with those with low staging (both P<0.05).

### Analysis of Efficacy-Related Influencing Factors

As shown in Table 16, the results of univariate analysis showed that gender and vitamin supplementation were factors affecting the reversal of atrophic gastritis, whereas consumption of preserved foods, inflammatory state of the gastric mucosa, and H. pylori infection were factors affecting the reversal of intestinal metaplasia (all P<0.05). As shown in Table 17, the results of multifactorial logistic regression analysis showed that females, vitamin supplementation (≥3 times/week), H. pylori negative or successful eradication, and mild inflammation (inflammation score of 1-2) were associated with increased efficacy of the Lamb stomach extract Vitamin B₁₂ preparation, and high consumption of preserved foods (≥3 times/week) was associated with decreased efficacy (all P<0.05).

TABLE 16
Univariate analysis of factors affecting the efficacy of lamb stomach extract Vitamin B₁₂ preparation [cases (%)]

| | | | Atrophic Gastritis | | | | Intestinal Metaplasia | | | |
|---|---|---|---|---|---|---|---|---|---|---|
| Relevant | | | | | x² | | | | x² | |
| Factor | | Case | Negative | Effective | Value | P Value | Negative | Effective | Value | P Value |
| Sex | | | | | 4.869 | 0.034 | | | 0.413 | 0.586 |
| Male | | 146 | 88(60.3) | 58(39.7) | | | 93(63.7) | 53(36.3) | | |
| Female | | 94 | 43(45.7) | 51(54.3) | | | 56(59.6) | 38(40.4) | | |
| Age (Year) | | | | | 0.126 | 0.786 | | | 1.605 | 0.212 |
| <50 | | 83 | 44(53.0) | 39(47.0) | | | 47(56.6) | 36(43.4) | | |
| N50 | | 157 | 87(55.4) | 70(44.6) | | | 102(65.0) | 55(35.0) | | |
| Blood Type | | | | | 3.280 | 0.519 | | | 1.099 | 0.896 |
| A | | 44 | 17(38.6) | 27(61.4) | | | 14(31.8) | 30(68.2) | | |
| B | | 47 | 20(42.6) | 27(57.4) | | | 18(38.3) | 29(61.7) | | |
| 0 | | 52 | 29(55.8) | 23(44.2) | | | 20(38.5) | 32(61.5) | | |
| AB | | 22 | 10(45.5) | 12(54.5) | | | 8(36.4) | 14(65.6) | | |
| Unknown | | 75 | 33(44.0) | 42(56.0) | | | 31(41.3) | 44(58.7) | | |
| BMI\(kg/m²) | | | | | 1.188 | 0.758 | | | 0.362 | 0.959 |
| <18.5 | | 17 | 10^{a} | 7^{a} | | | 11^{a} | 6^{a} | | |
| 18.5~23.9 | | 147 | 79(53.7) | 68(46.3) | | | 90(61.2) | 57(38.8) | | |
| 24.0~28.0 | | 66 | 35(53.0) | 31(47.0) | | | 41(62.1) | 25(37.9) | | |
| >28.0 | | 10 | 7^{a} | 3^{a} | | | 7^{a} | 3^{a} | | |
| Education | | | | | 2.139 | 0.175 | | | 1.761 | 0.211 |
| Above High school | | 155 | 90(58. 1) | 65(41.9) | | | 101(65.2) | 54(34.8) | | |
| High school or below | | 85 | 41(48.2) | 44(51.8) | | | 48(56.5) | 37(44.5) | | |
| Residue | | | | | 2.758 | 0.124 | | | 0.489 | 0.566 |
| Urban | | 165 | 96(58.2) | 69(41.8) | | | 100(60.6) | 65(39.4) | | |
| Countryside | | 75 | 35(46.7) | 40(53.3) | | | 49(65.3) | 26(34.7) | | |
| Family history of | | | | | 0.193 | 0.759 | | | 1.034 | 0.348 |
| stomach cancer | | | | | | | | | | |
| Yes | | 56 | 32(57.1) 1) | 24(42.9) | | | 38(67.9) | 18(32.1) | | |
| No | | 184 | 99(53.8) | 85(46.2) | | | 111(60.3) | 73(39.7) | | |
| History of | | | | | 0.039 | 1.000 | | | 1.717 | 0.260 |
| cholecystectomy | | | | | | | | | | |
| Yes | | 14 | 8^{a} | 6^{a} | | | 11^{a} | 3^{a} | | |
| No | | 226 | 123(54.4) | 103(45.6) | | | 138(61.1) | 88(38.9) | | |
| High blood pressure | | | | | 0.250 | 1.(XX) | | | 0.346 | 0.604 |
| Yes | | 43 | 23(53.5) | 20(46.5) | | | 25(58.1) | 18(41.9) | | |
| No | | 197 | 108(54.8) | 89(45.2) | | | 124(62.9) | 73(37.1) 1) | | |
| Diabetes | | | | | 0.565 | 0.584 | | | 0.312 | 1.000 |
| Yes | | 14 | 9^{a} | 5^{a} | | | 9^{a} | 5^{a} | | |
| No | | 226 | 122(54.0) | 104(46.0) | | | 140(61.9) | 86(38.1) | | |
| Non-steroid anti- | | | | | 1.160 | 0.379 | | | 1.061 | 0.367 |
| inflammatory drug | | | | | | | | | | |
| ≥3 | times/week | 23 | 15(65.2) | 8(34.8) | | | 12(52.2) | 11(47.8) | | |
| <3 | times/week | 217 | 116(53.5) | 101(46.5) | | | 137(63.1) | 80(36.9) | | |
| Metformin | | | | | 0.088 | 1.(K)0 | | | 0.019 | 1.(XX) |
| ≥3 | times/week | 10 | 5^{a} | 5^{a} | | | 6^{a} | 4^{a} | | |
| <3 | times/week | 230 | 126(54.8) | 104(45.2) | | | 143(62.2) | 87(37.8) | | |
| Statins | | | | | 0.569 | 0.534 | | | 0.135 | 0.832 |
| ≥3 | times/week | 26 | 16(61.5) | 10(38.5) | | | 17(65.4) | 9(34.6) | | |
| <3 | times/week | 214 | 115(53.7) | 99(46.3) | | | 132(61.7) | 82(38.3) | | |
| vitamins | | | | | 5.686 | 0.019 | | | 1.353 | 0.336 |
| ≥3 | times/week | 20 | 4(20.0) | 16(80.0) | | | 10(50.0) | 10(50.0) | | |
| <3 | times/week | 220 | 115(52.3) | 105(47.7) | | | 139(63.2) | 81(36.8) | | |
| cigarette smoking | | | | | 1.847 | 0.220 | | | 0.005 | 1.000 |
| ≥5 | pcs/d | 56 | 35(62.5) | 21(37.5) | | | 35(62.5) | 21(37.5) | | |
| <5 | pcs/d | 184 | 96(52.2) | 88(47.8) | | | 114(62.0) | 70(38.0) | | |
| Alcohol drinking | | | | | 0.731 | 0.405 | | | 0.346 | 0.604 |
| ≥100 | g/week | 43 | 26(60.5) | 17(39.5) | | | 25(58.1) | 18(41.9) | | |
| <100 | g/week | 197 | 105(53.3) | 92(46.7) | | | 124(62.9) | 73(37.1) | | |
| high salt diet | | | | | 0.700 | 0.424 | | | 1.984 | 0.172 |
| ≥3 | times/week | 90 | 46(51.1) | 44(48.9) | | | 61(67.8) | 29(32.2) | | |
| <3 | times/week | 150 | 85(56.7) | 65(43.3) | | | 88(58.7) | 62(41.3) | | |
| preserved food | | | | | 0.206 | 0.667 | | | 7.779 | 0.007 |
| ≥3 | times/week | 67 | 35(52.2) | 32(47.8) | | | 51(76.1) | 16(23.9) | | |
| <3 | times/week | 173 | 96(55.5) | 77(45.5) | | | 98(56.6) | 75(43.4) | | |
| overnight food | | | | | 0.451 | 0.575 | | | 0.093 | 0.776 |
| ≥3 | times/week | 74 | 38(51.4) | 36(48.6) | | | 47(63.5) | 27(36.5) | | |
| <3 | times/week | 166 | 93(56.0) | 73(44.0) | | | 102(61.4) | 64(38.6) | | |
| fried food | | | | | 0.780 | 0.423 | | | 3.391 | 0.071 |
| ≥3 | times/week | 49 | 24(49.0) | 25(51.0) | | | 36(73.5) | 13(26.5) | | |
| <3 | times/week | 191 | 107(56.0) | 84(44.0) | | | 113(59.2) | 78(40.8) | | |
| Fresh Vegetables | | | | | 0.609 | 0.510 | | | 0.021 | 0.893 |
| ≥250 | g/d | 99 | 57(57.6) | 42(42.4) | | | 62(62.6) | 37(37.4) | | |
| <250 | g/d | 141 | 74(52.5) | 67(47.5) | | | 87(61.7) | 54(38.3) | | |

| | | | | | | | | | |
|---|---|---|---|---|---|---|---|---|---|
| fruit | | | | 0.200 | 0.724 | | | 3.885 | 0.067 |
| ≥200/week | 202 | 109(54.0) | 93(46.0) | | | 120(59.4) | 82(40.6) | | |
| <200/week | 38 | 22(57.9) | 16(42.1) | | | 29(76.3) | 9(23.7) | | |
| Inflammation score | | | | 0.710 | 0.705 | | | 6.960 | 0.025 |
| No change | 161 | 89(55.3) | 72(44.7) | | | 96(59.6) | 65(40.4) | | |
| Increase | 27 | 16(59.3) | 11(40.7) | | | 23(85.2) | 4(14.8) | | |
| Decrease | 52 | 26(50.0) | 26(50.0) | | | 30(57.7) | 22(42.3) | | |
| Helicobacter pylori | | | | 0.247 | 0.679 | | | 4.844 | 0.033 |
| Positive | 26 | 13(50.0) | 13(50.0) | | | 11(42.3) | 15(57.7) | | |
| Negative | 214 | 118(55.1) | 96(44.9) | | | 138(64.5) | 76(35.5) | | |

Note:
^{a}The total number of cases is less than 20, so it is not expressed as a percentage.

TABLE 17
Multifactorial analysis affecting the efficacy of Vitamin
B₁₂ preparation composition of lamb stomach extracts

| Disease | Factor | Specific Ratio | 95% CI | P Value |
|---|---|---|---|---|
| Atrophic gastritis | Sex (female) | 1.798 | 1.055~3.064 | 0.031 |
| | Vitamin supplementation (≥3 | 3.730 | 1.197~11.627 | 0.023 |
| Intestinal | Consumption of large quantities of | 0.384 | 0.200~0.740 | 0.004 |
| metaplasia | preserved foods (≥3 times/week) | | | |
| | Helicobacter pylori negative or | 2.817 | 1.171~6.779 | 0.021 |
| | Mild inflammation (inflammation score | 4.631 | 1.480~14.493 | 0.008 |

As shown in Table 18, the results of stratified analysis showed that the differences in atrophy reversal rate and intestinal metaplasia reversal rate between the H. pylori positive eradication treatment group (76 cases) and the H. pylori negative uneradicated treatment group (164 cases) were not statistically significant (both P>0.05). The total effective reversal rate of the H. pylori positive eradication treatment group was higher than that of the H. pylori negative uneradicated treatment group, and the difference was statistically significant (χ²=5.388, P<0.05). The differences in the total effective reversal rate, atrophy reversal rate and enteroclastogenic reversal rate between patients with successful eradication and those with unsuccessful eradication within the H. pylori eradication treatment group were not statistically significant (P all>0.05). The total effective reversal rate, atrophy reversal rate and enteroclastogenic reversal rate of the patients with retest-negative versus retest-positive H. pylori-negative without eradication treatment group were statistically insignificant (P all>0.05). The total effective reversal rate, atrophy reversal rate and intestinal metaplasia reversal rate were not statistically significant when compared (all P>0.05).

TABLE 18
Effect of H. pylori eradication treatment on the efficacy
of lamb stomach extract Vitamin B₁₂ preparation [cases (%)].

| | | Atrophy reversal | Reversal rate of | Total effective |
|---|---|---|---|---|
| Groups | Cases | rate | intestinal metaplasia | reversal rate |
| Positive eradication treatment | 76 | 40(52.6) | 33(43.4) | 55(72.4)^{a} |
| group | | | | |
| Eradication success | 58 | 31(53.5) | 24(41.4) | 42(72.4) |
| Eradication failure | 18 | 9(50.0) | 9(50.0) | 13(72.2) |
| Negative untreated group | 164 | 69(42.1) | 58(35.4) | 93(56.7) |
| Retested Negative | 156 | 65(41.7) | 52(33.3) | 87(55.8) |
| Retested Positive | 8 | 4(50.0) | 6(75.0) | 6(75.0) |

Note:
^{a}P < 0.05 vs. negative untreated eradication group

As shown in Table 19, the proportion of patients with complete reversal of atrophy who consumed persevered foods was lower than that of patients with progression. The proportions of those who consumed fruits, had high OLGA staging, and were treated with H. pylori eradication were higher than those of patients with progression, and the differences were all statistically significant (χ²=9.701, 40.518, 4.575, and 4.023, all P<0.05). The proportions of alcohol consumption, consumption of preserved food, and high OLGA stage were lower in patients with complete reversal of intestinal metaplasia than in patients with progression, and the differences were all statistically significant (χ²=5.315, 3.958, 40.518, all P<0.05). Gender, age, family history of gastric cancer, smoking history, high salt diet, consumption of vegetables and OLGIM staging were not statistically significant differences between the atrophy and complete reversal of intestinal metaplasia group and the progression group (all P>0.05).

TABLE 19
Clinical characteristics of patients with complete reversal of atrophy and intestinal metaplasia versus those with progression

| | Atrophy | | | | Intestinal metaplasia | | | |
|---|---|---|---|---|---|---|---|---|
| | Complete | | | | Complete | | | |
| Clinical | reversal | Progressed | Statistical | P- | reversal | Progressed | Statistical | |
| Characteristic | (n = 35) | (n = 51) | value | value | (n = 35) | (n = 51) | value | P-value |
| Sex [cases (%)] | | | χ² = 0.400 | 0.527 | | | χ² = 2.246 | 0.134 |
| Male | 21(60.0) | 34(66.7) | | | 12^{a} | 30(58.8.5) | | |
| Female | 14(40.0) | 17(33.3) | | | 3^{a} | 21(41.2) | | |
| Age (years, x̅ ± s) | 54.73 ± 8.63 | 53.34 ± 7.80 | t = 0.068 | 0.945 | 52.64 ± 10.79 | 53.58 ± 7.70 | t = 1.226 | 0.228 |
| Family history of gastric | | | χ² = 0.075 | 0.783 | | | χ² = 0.402 | 0.715 |
| cancer [cases (%)] | | | | | | | | |
| Yes | 7(20.0) | 9(17.6) | | | 4^{a} | 12(23.5) | | |
| No | 28(80.0) | 42(82.4) | | | 11^{a} | 39(76.5) | | |
| History of smoking | | | χ² = 0.199 | 0.654 | | | χ² = 3.027 | 0.108 |
| [(Cases (%)] | | | | | | | | |
| Yes | 9(25.7) | 11(21.6) | | | 7^{a} | 12(23.5) | | |
| No | 26(74.3) | 40(78.4) | | | 8^{a} | 39(76.5) | | |
| History of alcohol | | | χ² = 1.236 | 0.266 | | | χ² = 5.315 | 0.036 |
| [(Cases (%)] | | | | | | | | |
| Yes | 6(17.1) | 14(27.5) | | | 8^{a} | 42(82.4) | | |
| No | 29(82.9) | 37(72.5) | | | 7^{a} | 9(17.6) | | |
| High-salt diet | | | χ² = 0.091 | 0.763 | | | χ² = 1.037 | 0.308 |
| [(Cases (%)] | | | | | | | | |
| Yes | 16(45.7) | 25(49.0) | | | 4^{a} | 21(41.2) | | |
| No | 19(54.3) | 26(51.0) | | | 11^{a} | 30(58.8) | | |
| Preserved food | | | χ² = 9.701 | 0.002 | | | χ² = 3.958 | 0.046 |
| [(Cases (%)] | | | | | | | | |
| Yes | 10(28.6) | 32(62.7) | | | 2^{a} | 21(41.2) | | |
| No | 25(71.4) | 19(37.3) | | | 13^{a} | 30(58.8) | | |
| Vegetables | | | χ² = 0.502 | 0.479 | | | χ² = 0.946 | 0.331 |
| [(Cases (%)] | | | | | | | | |
| Yes | 15(42.9) | 18(35.3) | | | 8^{a} | 20(39.2) | | |
| No | 20(57.1) | 33(64.7) | | | 7^{a} | 31(60.8) | | |
| Fruit [(Cases (%)] | | | χ² = 40.518 | 0.003 | | | χ² = 2.677 | 0.182 |
| Yes | 32(91.4) | 11(21.6) | | | 15^{a} | 43(84.3) | | |
| No | 3(8.6) | 40(78.4) | | | 0 | 8(15.7) | | |
| OLGA staging | | | χ² = 4.575 | 0.032 | | | χ² = 7.382 | 0.006 |
| [(Cases (%)] | | | | | | | | |
| High Stage | 11(31.4) | 7(13.7) | | | 2^{a} | 27(52.9) | | |
| Low Stage | 24(68.6) | 44(86.3) | | | 13^{a} | 24(47.1) | | |
| OLGIM staging | | | χ² = 0.840 | 0.359 | | | χ² = 0.317 | 1.000 |
| [(Cases (%)] | | | | | | | | |
| High Stage | 6(17.1) | 13(25.5) | | | 1^{a} | 6(11.8) | | |
| Low Stage | 29(82.9) | 38(74.5) | | | 14^{a} | 45(88.2) | | |
| Helicobacter pylori | | | χ² = 4.023 | 0.044 | | | χ² = 0.226 | 0.634 |
| infection and treatment | | | | | | | | |
| [(Cases (%)] | | | | | | | | |
| Positive (eradicated) | 12(34.3) | 8(15.7) | | | 6^{a} | 17(33.3) | | |
| Negative (not eradicated) | 23(65.7) | 43(84.3) | | | 9^{a} | 34(66.7) | | |

Note:
OLGA is the gastritis evaluation system; OLGIM is the gastritis evaluation system based on intestinal metaplasia.
^{a}The total number of cases is less than 20, so it is not expressed as a percentage

The results of the present invention showed that the overall effectiveness rate of the lamb stomach extract Vitamin B₁₂ preparation composition in the treatment of atrophy and intestinal metaplasia was 62.9%, which was comparable to the results of previous studies. In addition, the present invention, after risk stratification of CAG patients by the OLGA and OLGIM staging system, found that the lamb stomach extract Vitamin B₁₂ preparation composition was highly effective in patients with high OLGA and OLGIM staging (Stages III and IV), with up to about 70% of Stage IV patients. The treatment could reverse 54.5% (36/66) of patients with intestinal metaplasia from high OLGIM stage to low OLGIM stage, suggesting that the treatment with lamb stomach extract Vitamin B₁₂ preparation could reduce the risk of gastric cancer in patients with CAG.

Helicobacter pylori infection is the most important cause of gastric cancer, and the continuous activation of the inflammatory-cancer pathway and intracellular positive feedback of nuclear factor-κ B, signal transducer and activator of transcription 3 in the infected state is one of the most important risk factors for the irreversible progression of gastric mucosal lesions. Eradication of H. pylori can effectively block the inflammation-cancer pathway and significantly reduce the incidence of gastric cancer in the population. After eradication of H. pylori, intestinal metaplasia remains an important risk factor for gastric cancer development and progression. Several studies and meta-analyses have shown that eradication of H. pylori can only reverse gastric mucosal atrophy, but not intestinal metaplasia. Therefore, in the Correa model of gastric cancer development, intestinal metaplasia is often regarded as an irreversible point of gastric mucosal lesion. The present invention and other previous studies have confirmed that gastric mucosal atrophy and intestinal metaplasia can be reversed with drug intervention, indicating that it is not appropriate to regard intestinal metaplasia as an irreversible point of gastric mucosal lesion. Some studies have also pointed out that after 5-10 years of follow-up, 33.9% (75/221) and 44.4% (63/142) of the H. pylori eradication group in the gastric sinus and gastric body, respectively, had reversal of intestinal metaplasia, suggesting that eradication of H. pylori is able to reverse atrophy and intestinal metaplasia to a certain degree, but the reversal effect is weaker and requires a longer accumulation of temporal effects. The results showed that lamb stomach extract Vitamin B₁₂ preparation was effective in reversing atrophy and intestinal metaplasia with or without H. pylori infection, and eradication treatment increased the overall reversal efficiency of Vitamin B₁₂ preparation of lamb stomach extract on atrophy and intestinal metaplasia.

In conclusion, based on the eradication of H. pylori, treatment with lamb stomach extract Vitamin B₁₂ preparation has a better reversal of atrophy and intestinal metaplasia and may reduce the risk of gastric cancer in patients with CAG. Treatment with lamb stomach extract Vitamin B₁₂ preparation should be recommended for high-risk (OLGA, OLGIM stage III and IV) CAG patients.

### Example 8 Reversal of Chronic Atrophic Gastritis with Severe Intestinal Metaplasia by Lamb Stomach Extract Vitamin B₁₂ Preparation

The patient, a 52-year-old woman, was admitted to the outpatient clinic of the Department of Gastroenterology of Xijing Hospital of Air Force Medical University on Mar. 13, 2018, for “abdominal distension, belching and other epigastric discomfort, which had been aggravated for 1 month.”

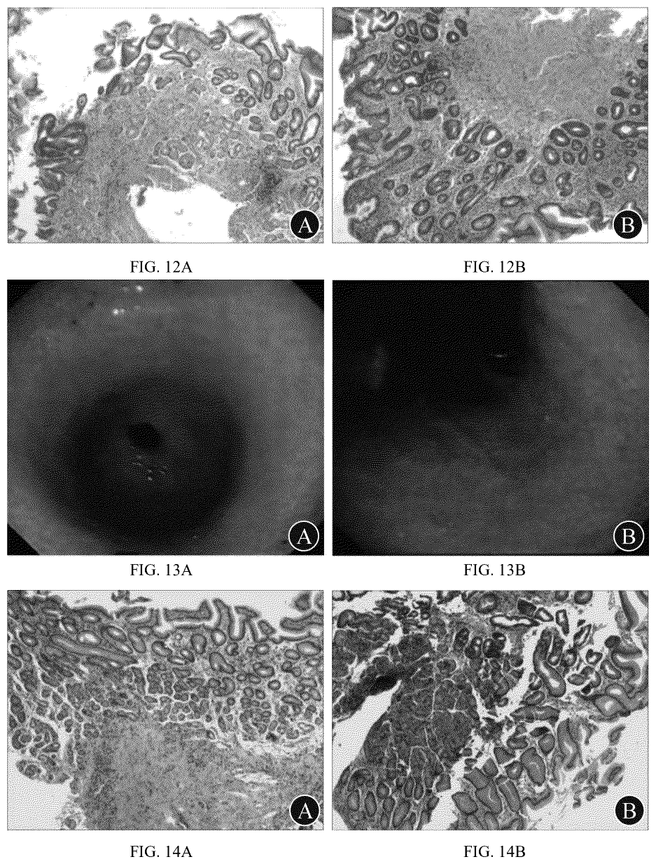

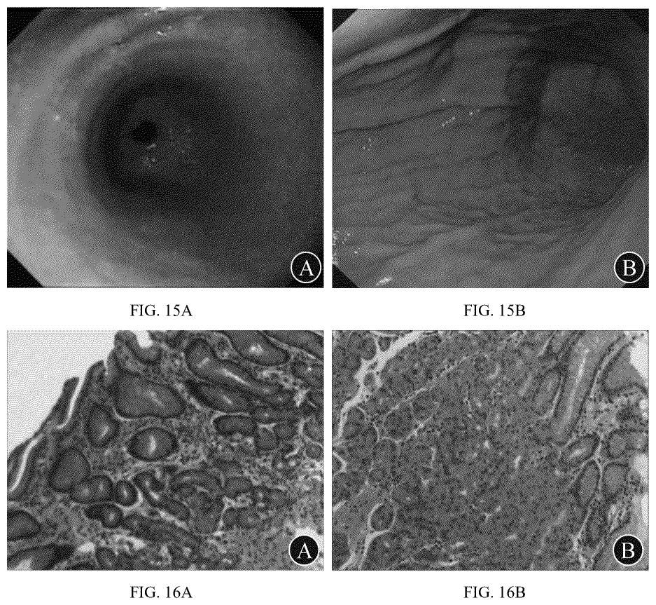

13C-UBT suggested negative, gastroscopy results as shown in FIGS. 11A-11B, suggesting that the gastric mucosa is less smooth, obvious in the gastric body, the color phase is obvious, submucosal vascular lines are seen through, scattered points of flaky erosion and old hemorrhagic spots; the body of the stomach, the angle of the stomach and the gastric sinus part of the grayish-white microembolism, part of the surface of the slightly depressed, reddened, narrow-band endoscopic observation to see the bright blue cristae-like changes, mostly intestinal metaplasia. Samples were taken from the upper part of the gastric body on the side of the big and small curves, the gastric angle and the side of the big and small curves of the gastric sinus, respectively, and the tissues were soft, peristalsis was normal, and the mucus lake was slightly turbid. Pathological findings, as shown in FIGS. 12A-12B, suggested that the mucosa of the upper part of the gastric body on the side of the greater and lesser curvature showed moderate chronic inflammation, severe atrophy, and severe intestinal metaplasia; the mucosa of the gastric angle and the side of the greater and lesser curvature of the gastric sinus department showed moderate chronic inflammation, moderate atrophy, and mild intestinal metaplasia. Operative link on gastritis assessment (OLGA)/operative link on gastritis assessment based on intestinal metaplasia (OLGIM) Staging was stage IV/III. Diagnosis: chronic atrophic gastritis with severe intestinal metaplasia. The patient was accompanied by epigastric discomfort such as abdominal distension and belching, and was treated with oral lamb stomach extract Vitamin B₁₂ preparation. After six months of treatment, the 13C-UBT was reviewed on Dec. 11, 2019 and was negative. Gastroscopy results (FIGS. 13A-13B) suggests that the large curvature side of the lower part of the responsible portal can be seen flaky mucosa shallow depression, the bottom is not mossy, the edge of the mucosa is still smooth, slightly congested, and the tissue at the sampling site is soft; the lower part of the lower part of the body of the stomach can be seen in the side of the small curvature of the flaky mucosa is slightly elevated, with the tip of the vesicles, and the tissue at the sampling site is soft; the rest of the gastric mucosa is still smooth, soft, and reddish-white color, the sinus part of the scattered points of the flaky mucous membrane erythema, peristaltic movement can be, and mucus lakes are slightly turbid.12.12.2019 Pathology biopsy results on December 11 are shown in FIGS. 14A-14B, suggesting moderate chronic inflammation of the mucosa of the lower part of the gastric body, with mild acute activity, moderate atrophy, moderate intestinal metaplasia, and microconvex epithelial metaplasia; chronic inflammation of the cardia mucosa with acute activity, and microconvex epithelial metaplasia with moderate intestinal metaplasia; mild chronic inflammation of the sinus and antrum mucosa, with mild acute activity, and mild-moderate atrophy; and mild chronic inflammation of the mucosa of the upper part of the gastric body. OLGA/OLGIM Staging is stage III/II. Diagnosis: chronic atrophic gastritis with intestinal metaplasia. The patient's gastric mucosal atrophy and intestinal metaplasia were reduced compared to the previous level, and he was instructed to continue oral treatment with lamb stomach extract Vitamin B₁₂ preparation, and to repeat the examination after half a year. On Mar. 30, 2021, the examination was repeated with negative 13C-UBT. Gastroscopy results as shown in FIGS. 15A-15B, suggesting that the cardia local mucosal congestion; the remaining gastric mucosa is soft and not smooth, to the small curvature of the body of the stomach side is obvious, the color of red and white is obvious, submucosal vascular lines see through, peristalsis is normal, the mucus lake is slightly turbid. Samples were taken from the upper part of the upper curvature of the gastric body, the angle of the stomach and the curvature of the gastric antrum, respectively, and the tissue was soft. Pathologic biopsy results (FIGS. 16A-16B) suggested moderate chronic inflammation of the mucosa of the gastric sinus and gastric angle, mild acute activity, mild atrophy, and small concave epithelial metaplasia; the mucosa of the gastric body was mildly chronic inflammation. The OLGA/OLGIM staging was stage I/stage 0. Diagnosis: chronic atrophic gastritis, carditis. After 1.5 years of treatment with lamb stomach extract Vitamin B₁₂ preparation, the patient's gastric mucosa OLGA stage was reduced from stage IV to stage I, and OLGIM stage was reduced from stage III to stage 0. It is suggested that lamb stomach extract Vitamin B₁₂ preparation can effectively reduce the OLGA and OLGIM stages of patients with CAG with intestinal metaplasia, and may reduce the risk of gastric cancer in the patients.

The foregoing is a preferred embodiment of the present invention, and it should be mentioned that for a person of ordinary skill in the art, a number of improvements and embellishments may be made without departing from the principles mentioned in the present invention, and these improvements and embellishments should also be considered as the scope of protection of the present invention.

What is claimed is:

1. A lamb stomach extract Vitamin B₁₂ preparation composition, wherein the lamb stomach extract Vitamin B₁₂ preparation composition is composed of a lamb stomach extract and Vitamin B₁₂, the lamb stomach extract has a chymosin activity of >40 U/g and a pepsin activity of >15 U/g, and a weight ratio of the lamb stomach extract and the Vitamin B₁₂ is 100000:1-4.5.

2. (canceled)

3. The lamb stomach extract Vitamin B₁₂ preparation composition according to claim 1, wherein the weight ratio of the lamb stomach extract and the Vitamin B₁₂ is 100000:1, 100000:2, 100000:3, 100000:4, or 100000:4.5.

4. The lamb stomach extract Vitamin B₁₂ preparation composition according to claim 1, wherein the chymosin activity is >45 U/g and the pepsin activity is >20 U/g; or the chymosin activity is >55 U/g and the pepsin activity is >20 U/g.

5. The lamb stomach extract Vitamin B₁₂ preparation composition according to claim 1, wherein the lamb stomach extract has a relative molecular weight of 10,000 to 45,000.

6. The lamb stomach extract Vitamin B₁₂ preparation composition according to claim 1, wherein a ratio of the chymosin activity to the pepsin activity is from 2.5 to 5.1.

7. The lamb stomach extract Vitamin B₁₂ preparation composition according to claim 1, further comprising a pharmaceutically acceptable additive.

8. The lamb stomach extract Vitamin B₁₂ preparation composition according to claim 7, wherein the pharmaceutically acceptable additive is one or more of an excipient, a binder, a diluent, a flow aid, a flavoring agent, an antioxidant, and a bacteriostatic agent.

9. The lamb stomach extract Vitamin B₁₂ preparation composition according to claim 1, wherein a dosage form of the lamb stomach extract Vitamin B₁₂ preparation composition is a capsule, a tablet, a suspension, or a granule.

10. A manufacture method of a medicament for reversing an atrophy of gastric mucosa, comprising using a lamb stomach extract Vitamin B₁₂ preparation composition.

11. A manufacture method of a medicament for a treatment of moderate or severe chronic atrophic gastritis, comprising using a lamb stomach extract Vitamin B₁₂ preparation composition.

12. A manufacture method of a medicament for a treatment of an intestinal metaplasia, comprising using a lamb stomach extract Vitamin B₁₂ preparation composition.

13. The manufacture method according to claim 12, wherein the intestinal metaplasia is an intestinal metaplasia accompanied with chronic atrophic gastritis.

14. A manufacture method of a medicament for a treatment of gastric intraepithelial neoplasia, comprising using a lamb stomach extract Vitamin B₁₂ preparation composition.

15. A manufacture method of a medicament for a prevention of a gastric cancer, comprising using a lamb stomach extract Vitamin B₁₂ preparation composition.

16. The manufacture method according to claim 11, wherein the treatment is an effective treatment.

17. The manufacture method according to claim 11, wherein the treatment is a reversal treatment.

18. The manufacture method according to claim 10, wherein a subject for an administration is a Helicobacter pylori-negative patient.

19. The manufacture method according to claim 11, wherein the treatment is carried out through an antioxidant pathway.

20. The manufacture method according to claim 11, wherein the treatment, reversal is carried out by regulating intestinal flora dysbiosis.

21. The lamb stomach extract Vitamin B₁₂ preparation composition according to claim 8, wherein the lamb stomach extract has the chymosin activity of not less than 45 U/g and the pepsin activity of not less than 20 U/g; or the chymosin activity of not less than 55 U/g and the pepsin activity of not less than 20 U/g; the lamb stomach extract Vitamin B₁₂ preparation composition is administered to a patient at 330 U, 3 times/d; and the lamb stomach extract has a relative molecular weight of 10,000 to 45,000.
